(19) United States
(12) Patent Application Publication
(10) Pub. No.: US 2024/0100234 A1
(43) Pub. Date: Mar. 28, 2024

(54) SYSTEM AND METHODS TO ENHANCE CHEMOTHERAPY DELIVERY AND REDUCE TOXICITY

(71) Applicant: Sigyn Therapeutics, San Diego, CA (US)

(72) Inventor: James A. Joyce, San Diego, CA (US)

(21) Appl. No.: 18/373,829

(22) Filed: Sep. 27, 2023

Related U.S. Application Data
(60) Provisional application No. 63/410,764, filed on Sep. 28, 2022.

(51) Int. Cl. A61M 1/36

(52) U.S. Cl. CPC A61M 1/3615

(57) ABSTRACT

Despite therapeutic advances in the field of oncology, treatment toxicity, drug resistance, and inadequate tumor site delivery restrict the benefit of cancer chemotherapy regimens. Disclosed are extracorporeal devices comprising adsorbent components that are suitable for treating a subject prior to and following administration of a dose of a chemotherapy drug, wherein the devices are configured to remove circulating factors comprising a chemotherapy drug from blood or plasma. Also disclosed herein are methods of reducing the bloodstream presence of circulating factors that mediate drug resistance, drug toxicity, and cancer metastasis, wherein circulating factors include without limitation, tumor-derived and/or chemotherapy-induced extracellular vesicles or exosomes.

### CROSS-REFERENCE TO RELATED APPLICATIONS

The present application claims priority to and benefit from U.S. Provisional Patent Application No. 63/410,764, entitled SYSTEM AND METHODS TO ENHANCE CHEMOTHERAPY DELIVERY AND REDUCE TOXICITY and filed on Sep. 28, 2022, the entire contents of which are hereby expressly incorporated by reference.

### FIELD OF INVENTION

The invention relates to extracorporeal blood purification systems for removing chemotherapy drugs and vesicles that are present in a bodily fluid, and methods of use for treating a subject with cancer. Specifically, the invention provides medical devices and methods of use in oncology practice to improve the efficacy and safety of chemotherapeutic agents.

### BACKGROUND

Cancer is the second leading cause of death in the United States. Despite therapeutic advances in oncology, challenges such as treatment toxicity and drug resistance restrict the benefit of cancer chemotherapy regimens. High doses of chemotherapy have been shown to be effective in the treatment of various types of cancer and may be useful in overcoming resistance to certain chemotherapy drugs. It is estimated that 90% of failures of chemotherapy occur during advanced metastatic disease stages and are related to drug resistance. Following the administration of a particular drug, large numbers of tumor cells become resistant to the drug. Additionally, due to the distribution of many chemotherapeutic agents to organs and tissues throughout the body, toxicity, many adverse events are caused by these drugs. The use of high dose chemotherapy has been limited, while even lesser doses can lead to adverse events that underlie treatment discontinuation, delays and interruptions that compromise the treatment outcomes for many patients. Therefore, treatment approaches that limit systemic exposure to chemotherapy drugs while maximizing effects on target tumors are sought.

Studies have elucidated the cellular and molecular mechanisms by which tumors evade the actions of chemotherapy drugs to mediate primary/intrinsic and acquired drug resistance. In cancer patients' plasma, circulating vesicles known as extracellular vesicles (e.g., exosomes) that primarily originate from tumor cells, harbor protein and nucleic acid cargo that confers drug resistance. These vesicles are immensely concentrated in the bloodstream of patients with cancer (10× to >500× higher concentrations than in healthy individuals). These vesicles also encapsulate chemotherapy drugs and serve as vehicles for the efflux of drug molecules out of tumor cells, thereby reducing the treatment effects of the drug. Moreover, the effluxed drug can then be transported to off-target locations in the body. The actions of extracellular vesicles in transporting malignancy-associated factors and as purveyors of toxic drugs remain unaddressed in oncology.

### SUMMARY

The invention provides extracorporeal devices and methods for clearance of targets from the circulatory system that underlie chemotherapy treatment challenges in oncology. The invention stems from the need for methods to reduce the systemic levels of chemotherapy drugs and drug-associated factors in the blood to improve treatment outcomes and to improve quality of life for patients with cancer who are receiving a chemotherapy treatment regimen. The devices and methods of the present invention are designed to address one or more of the following targets: a chemotherapy drug, a metabolite of a chemotherapy drug, an extracellular vesicle such as an exosome, a molecule or drug that is associated with or contained within an extracellular vesicle, or portions thereof. In preferred embodiments, the devices and methods of the present invention bind or capture a plurality of targets from blood or plasma.

The methods of the present invention can be practiced using one or more extracorporeal blood purification devices having either identical or different physical structures and adsorbent compositions. In a preferred embodiment, the methods include two extracorporeal blood purification devices for treating a subject with cancer for the purpose of reducing the amounts of deleterious molecules or compounds from blood or plasma. In certain embodiments, a first extracorporeal device is used for clearing targets from blood or plasma from a subject prior to administration of a chemotherapy drug, wherein the targets of interest are induced or produced by a tumor or by other disease-affected cells in the subject, which may be referred to herein as “pre-chemotherapy target molecules or compounds”. The targets may include exosomes that have deleterious roles involving interference with the activity of the chemotherapy drug, mediating drug resistance, and spreading malignant cargo including proteins and nucleic acids from a tumor and/or from disease-affected cells. In certain embodiments, a chemotherapy drug is then administered to the subject and, following tissue distribution of the drug, an extracorporeal method is then performed using a second device for the purpose of clearing or reducing deleterious targets from blood or plasma (i.e., post-chemotherapy target molecules or compounds), which preferably include targets that result from or are incited by the chemotherapy drug administration.

In certain embodiments, the second device removes a fraction of blood or plasma that may include a chemotherapy drug and/or a metabolite of a chemotherapy drug, wherein a metabolite is a by-product of drug metabolism but exerts cytotoxic activity and/or mediates adverse events in a subject. In other embodiments, the second device removes exosomes that are carriers of chemotherapy drug and other tumor resistance factors. In other preferred embodiments, the disclosed methods provide for the simultaneous removal of exosomes, chemotherapy drug, and one or more metabolites of a chemotherapy drug by the second device.

In certain embodiments, more than one chemotherapy drug and/or the metabolites thereof are cleared or reduced from blood or plasma by an extracorporeal device of the invention. In some embodiments, methods using the second device in the post-chemotherapy period can be performed to reduce treatment toxicity by reducing the amount of circulating chemotherapeutic drug agents that are not delivered to the tumor site. In some embodiments, methods using the second device are applied to reduce the bloodstream presence of chemotherapy-induced targets such as exosomes that promote the spread of cancer metastasis. In other embodiments, the extracorporeal methods using a first device are performed to augment the benefits of treatment with a second device by facilitating increased removal of exosomes that may compete for target site binding or otherwise adversely impact the performance of a second device for reducing toxic drug levels. The first extracorporeal device and the second extracorporeal device may include the same adsorbent(s), wherein the adsorbent composition is configured for adsorption or binding to heterogeneous molecules and/or compounds. Alternatively, the adsorbent composition in the first and second extracorporeal devices may differ with respect to the adsorbent composition and/or adsorbent concentrations to accommodate the efficient removal of distinctive targets by the devices.

In another aspect, the devices disclosed herein are utilized in conjunction with conventional methods of therapy that are known to one skilled in the art in oncology, including standard of care chemotherapy treatment regimens. In certain embodiments, a chemotherapy drug includes one or more of the following: an antimetabolite, an antimicrotubular agent, an antibiotic, an anthracycline, an alkylating agent, an antibody-drug conjugate, or a drug metabolite thereof. In some embodiments, a chemotherapy drug may include a liposomal drug formulation or another nanocarrier-based drug formulation.

In yet another aspect, a method for reducing the incidence or severity of chemotherapy-associated adverse events that often lead to dose delays and modifications and therapy interruptions or discontinuation is provided. Advantageously, the methods are also useful for addressing drug resistance and malignancy-promoting factors carried by extracellular vesicles (e.g., exosomes), which may include cancer-derived or disease-related molecules, proteins, nucleic acids, lipids, glycoproteins, glycolipids or fragments or portions thereof. In certain embodiments, the methods disclosed herein are practiced prior to and/or following the administration of a dose of a chemotherapy drug to remove extracellular vesicles from blood or a blood component of a subject in need thereof. Accordingly, desired outcomes of the disclosed methods include improvement in the activity of a chemotherapy drug against a tumor, enhancement of drug efficacy, and mitigation of drug toxicity.

A method for improving the safety and efficacy of a chemotherapy drug is disclosed. The method may include:
a) introducing blood or plasma from a subject into a first extracorporeal device comprising an adsorbent, wherein the blood or plasma comprises an amount of exosomes;
b) contacting the blood or plasma with the adsorbent in the first extracorporeal device for a time sufficient to allow exosomes present in blood or plasma to bind to the adsorbent;
c) reintroducing the blood or plasma into the subject, wherein the blood or plasma obtained after (b) has a reduced amount of exosomes as compared to the blood or plasma of the subject prior to (b);
d) administering a chemotherapy drug to the subject;
e) introducing blood or plasma from the subject into a second extracorporeal device having an adsorbent, wherein the blood or plasma includes an amount of exosomes and chemotherapy drug;
f) contacting the blood or plasma with the adsorbent in the second extracorporeal device for a time sufficient to allow exosomes and chemotherapy drug present in blood or plasma to bind to the adsorbent; and
g) reintroducing the blood or plasma into the subject, wherein the blood or plasma obtained after (f) is measured to have a reduced amount of exosomes and chemotherapy drug as compared to the blood or plasma of the subject prior to (f).

In certain embodiments, treatment of a subject with an extracorporeal device is initiated through access to a patient's circulatory system, wherein the device is connected through the extracorporeal lines of the catheter to the subject's circulatory system. Access to the circulatory system may be obtained from arterial access or venous access. In one embodiment, access is obtained through the insertion of a central venous catheter into a patient.

In certain embodiments, an extracorporeal device is compatible for use with one or more commercially available, industry standard blood processing systems for performing hemodialysis, apheresis, continuous renal replacement therapy (CCRT), and therapeutic plasma exchange (TPE), to filter or remove one or more target factors from blood or a blood component. In some embodiments, an extracorporeal device disclosed herein is used in conjunction with an apheresis system. The apheresis process involves separation of plasma from the cellular fraction of whole blood of a subject, for example, using centrifugation, a membrane filter and/or other mechanisms. As a non-limiting example, a Spectra Optia® Apheresis System (Terumo BCT) may be used. The subject is connected to the apheresis system by obtaining vascular access, for example, using a central venous catheter such as a dual lumen indwelling catheter that is placed into the jugular or carotid vein of the subject.

An extracorporeal device of the present invention can be integrated into the extracorporeal circuit downstream from where the plasma is separated and into a secondary plasma device position of the circuit in accordance with the manufacturer's operating instructions for the apheresis machine. The subject's plasma is then recirculated through the extracorporeal device whereby the plasma passes through a substrate, which may comprise an adsorbent component, for the capture of one or more targets from the plasma. Flow rates can be adjusted within specific parameters that are suitable for the extracorporeal device in the circuit. The plasma is then pumped out of the system such that the treated plasma or a portion thereof is recombined with the separated cells and then is reintroduced into the circulatory system of the subject. In certain embodiments, the duration of an apheresis procedure is between 1 hour and 4 hours. In other embodiments, the treatment procedure is performed by processing between 1 and 4 plasma volumes of the subject. In preferred embodiments, 1.5 to 3 plasma volumes are processed. In a preferred embodiment, an apheresis procedure is performed prior to delivery of a dose of chemotherapy to the subject (e.g., on the same day and/or a day prior to chemotherapy administration) and/or following chemotherapy administration (e.g., on day 1 post-drug and/or on subsequent days).

In certain embodiments, an extracorporeal device is a cartridge that comprises a housing, an inlet port that receives a blood or a blood component (e.g., plasma), a chamber or compartment configured to receive the blood component from the inlet, and an outlet port configured to pass the treated blood or blood component out of the cartridge. The housing may be formed from plastic and/or other materials; non-limiting examples include polypropylene, polystyrene, polycarbonate, or Makrolon™ polycarbonate. The end caps may be affixed or coupled to the housing. In certain embodiments, the end caps terminate at an inlet and/or an outlet. An inlet and/or an outlet may be configured to be fluidically connected to tubing sets for apheresis. The length and diameter of the housing may be selected to optimize the flow dynamics of blood or plasma through the device. In certain embodiments, a chamber comprises one or a plurality of adsorbents that are configured to bind to a chemotherapy drug or a metabolite thereof, or an exosome.

In some embodiments, an extracorporeal device includes a hollow fiber membrane (which may interchangeably be referred to as a “filter”) disposed within a cartridge housing, wherein the hollow fiber membrane is comprised of a plurality of hollow fibers having fiber walls and a plurality of pores. In certain embodiments, the pores are sized and configured to allow blood or plasma components as small as 0.5 nm and as large as 200 nm to pass through the walls of the hollow fibers into an extraluminal space. In some embodiments, the hollow fiber membranes extend the length of the device. As whole blood or plasma enters the device, blood or plasma components with diameters less than 200 nm pass through the plurality of pores into an extraluminal space. In contrast, cells and blood or plasma components having diameters greater than about 200 nm in size are blocked from entering the extraluminal space and instead pass through the lumen of the hollow fibers. In preferred embodiments, the extraluminal space may include one or a plurality of adsorbents. As whole blood or plasma is filtered through the device, the plasma components that have accessed the extraluminal space encounter the one or plurality of adsorbents that positioned inside the housing and outside the hollow fiber filter. The targets are exposed to adsorbents in the extraluminal space such that the targets are bound, captured, sequestered, and/or adsorbed by the adsorbents, thereby reducing the amount of the target in the plasma. The device also may have an inlet port for receiving unfiltered (or untreated) blood or plasma and an outlet port for returning the treated blood or plasma, which has been depleted of targets, back to the circulatory system of the subject.

The extracorporeal devices may include one or more adsorbents for binding and capturing biological and non-biologic targets present in blood or plasma. The invention provides devices that can be applied both prior to and following the administration of chemotherapy drug to a subject. The invention also provides extracorporeal devices that satisfy the need to remove toxic chemotherapy drug from the circulation including free drug in circulation as well as drug component that is sequestered in and transported by extracellular vesicles such as exosomes.

The methods of the invention provide extracorporeal devices having adsorbents. The adsorbents preferably include activated carbon or least one non-ionic exchange resin or a combination thereof. More preferably, the extracorporeal methods of the invention provide adsorbents comprising activated carbon, a non-ionic aliphatic ester resin and a non-ionic polystyrene divinyl benzene resin. In some embodiments, the activated carbon has a microporous region with pore sizes less than 100 Angstroms, a mesoporous region with pore sizes between 100 and 1,000 Angstroms, and a macroporous region with pore sizes greater than 1,000 Angstroms. In one aspect, a non-ionic aliphatic ester resins have an average surface area of approximately 500 m²/g, an average pore size of 300-600 Angstroms, and a particle diameter of approximately 560 microns. In another aspect, a non-ionic polystyrene divinyl benzene resin has an average surface area of approximately 700 m²/g, an average pore size of 300 Angstroms, and mean particle diameter from approximately 35 microns to approximately 120 microns. In yet another aspect, a non-ionic polystyrene divinyl benzene resin has an average surface area of approximately 600 m²/g, an average pore size of 100-400 Angstroms, and mean particle diameter from approximately 300 microns to approximately 500 microns. In other embodiments, the extracorporeal methods apply adsorbents comprising an ion exchange resin, which may be included in an adsorbent composition also comprising activated carbon and/or at least one non-ionic exchange resin.

In one aspect of the invention, an extracorporeal device is provided. The extracorporeal device may include:
a) an inlet port for receiving unfiltered blood or plasma from a subject;
b) a compartment having a plurality of hollow fibers, wherein the hollow fibers have pore sizes of approximately 200 nm for passage of a plasma component such as a chemotherapy drug and/or exosomes;
c) an extraluminal space having an adsorbent composition, wherein the adsorbents may be activated carbon or at least one non-ionic exchange resin or both for contacting and binding the plasma component to form filtered blood or plasma; and
d) an outlet for returning filtered blood or plasma back to the subject.

In certain embodiments, the extracorporeal device can be utilized for treatment of a subject prior to or following the administration of a chemotherapy drug or at both time points. In certain embodiments, the adsorbent composition binds to a chemotherapy drug comprising a liposomal drug formulation.

Advantageously, embodiments of the invention provide devices and methods for simultaneous clearance of extracellular vesicles or exosomes and chemotherapy drug from blood or a blood component of a subject. The methods include filtering the blood or plasma through an extracorporeal device prior to and following administration of a chemotherapy drug or both, to achieve removal of deleterious disease-related and drug-induced extracellular vesicle. Populations as well as toxic chemotherapy drug. Certain embodiments provide extracorporeal methods for the removal of both “free” chemotherapy drug and exosome-encapsulated chemotherapy drug in circulation where the latter is not typically accounted for in conventional blood measurements of drug levels.

### DETAILED DESCRIPTION

The detailed description set forth below is intended as a description of various configurations of the subject technology and is not intended to represent the only configurations in which the subject technology may be practiced. The detailed description includes specific details for the purpose of providing an understanding of the subject technology. It will be apparent to those skilled in the art that the subject technology may be practiced without these specific details.

### Definitions

When describing an absolute value of a characteristic or property of a thing or act described herein, the terms “substantial,” “substantially,” “essentially,” “approximately,” “about” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

Unless defined otherwise, the technical terms used herein have the same meaning as is commonly understood by one of skill in the art.

As used herein, the term “subject” means any vertebrate organism having a circulatory system. In preferred embodiments, a subject is a mammal. A subject may be a human being. In preferred embodiments, a subject is a human patient. In certain embodiments, a subject has been diagnosed with and/or has symptoms related to the presence of one of more of the following types of cancer: melanoma (e.g., metastatic malignant melanoma), renal cancer, prostate cancer (e.g., hormone refractory prostate adenocarcinoma), breast cancer, colon cancer and non-small cell lung cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. Embodiments of the invention are applied for the treatment of a subject with metastatic cancer. In other embodiments, a subject has been diagnosed or has symptoms of an autoimmune disorder or a blood disorder. As used herein, the terms “treatment”, “therapy”, or “therapeutic” refer to an approach for obtaining beneficial results in the disease condition or symptoms of a subject, for example, using a drug or a device. Beneficial results may be obtained in clinical trials or from other clinical experiences and may include alleviation of symptoms, diminishing disease, stabilizing disease, preventing a disease from spreading, delaying disease progression, inducing remission, diminishing disease severity or slowing its recurrence, and/or affording improvements in a subject's quality of life. In the context of the invention, treatment methods comprise administering a therapeutically effective amount of an active agent or the use of a therapeutic device (e.g., an extracorporeal device). As used herein, the term “administration” of a drug may consist of administration of a single dose of said drug or it may comprise a series of administrations. As used herein, administration of therapy with a device may comprise a single treatment session or multiple treatment sessions with said device. Treatment with a drug or a device may be performed in an amount, for a duration, or using the number of repeat doses or treatment sessions that are sufficient to treat the patient.

As used herein, the terms “chemotherapy regimen”, “drug regimen”, or “treatment regimen” refer to a treatment plan wherein the drug(s) to be used, their dosage, and frequency and duration of treatments are defined for a subject and/or for a particular type of cancer. By way of example, a treatment regimen for a subject with multiple myeloma may involve administration bortezomib at 1.3 mg/m² as an intravenous bolus on days 1, 4, 8, and 11, every 3 weeks. On day 4 following bortezomib, Doxil® (i.e., a formulation of liposomal doxorubicin) is administered at a dose of 30 mg/m² intravenously over 60 minutes on day 4 of each 21-day cycle for eight cycles or until disease progression or unacceptable toxicity. The methods disclosed herein may be applied prior to and/or following the administration of one or more doses of a chemotherapy drug during a treatment regimen. As a non-limiting example, the claimed methods may be employed prior to and/or following the intravenous or oral administration of a drug in each cycle of a treatment regimen. In the treatment regimen example provided, the extracorporeal methods may be applied for on day 4 of the regimen prior to a Doxil® dose given the same day, on days 5-6 of the treatment regimen post-Doxil® dosing, and repetition of this treatment schedule for the eight-cycle treatment regimen.

In certain embodiments, a chemotherapy drug is an antimetabolite. In certain embodiments, an antimetabolite comprises one or more of the following types of compounds: a cytidine analog, a folate antagonist, a purine analog, or a pyrimdine analog. In some embodiments, the chemotherapeutic agent comprises one or more of the following drugs: 5-fluorouracil (5-FU), 6-mercaptopurine, 5-azacytidine/azacitidine, decitabine, capecitabine, cladribine, nelarabine, clofarabine, cytarabine, floxuridine, fludarabine, gemcitabine, methotrexate, pemetrexed, pentostatin, pralatrexate, hydroxyurea, thioguanine, or trifluridine/tipiracil combination. In certain embodiments, an antimetabolite is present in an extracellular vesicle or an exosome.

In another embodiment, a chemotherapy drug is an antimicrotubular agent. In certain embodiments, an antimicrotubular agent is comprised of one or more of the following types of compounds: a topoisomerase II inhibitor, a topoisomerase I inhibitor, a taxane, or a vinca alkaloid (i.e., a plant alkaloid). In certain embodiments, a topoisomerase II inhibitor comprises one or more of the following drugs: doxorubicin, liposomal doxorubicin, daunorubicin, idarubicin, mitoxantrone, etoposide, or teniposide. In preferred embodiments, a chemotherapeutic agent comprises doxorubicin, also known as doxorubicin hydrochloride, hydroxydaunorubicin, Adriamycin PFS, Adriamycin RDF, or Rubex. In certain embodiments, a topoisomerase I inhibitor comprises one or more of the following drugs: irinotecan, irinotecan liposomal or topotecan. In certain embodiments, a taxane comprises one or more of the following drugs: paclitaxel, nab-paclitaxel, docetaxel, cabazitaxel, vinblastine, vincristine, vincristine liposomal, or vinorelbine. In certain embodiments, a vinca alkaloid comprises one or more of the following drugs: vinblastine, vincristine, or vinorelbine. In certain embodiments, an antimicrotubular agent is present in an extracellular vesicle or an exosome.

In certain embodiments, a chemotherapy drug comprises an alkylating agent. In certain embodiments, an alkylating agent comprises one or more following drugs: altretamine, bendamustine, busulfan, carboplatin, chlorambucil, cisplatin, cyclophosphamide, dacarbazine, ifosfamide, mechlorethamine, melphalan, oxaliplatin, procarbazine, temozolomide, thiotepa, or trabectedin. In certain embodiments, an alkylating agent is a nitrosourea. In certain embodiments, a nitrosourea comprises carmustine, lomustine or streptozocin. In certain embodiments, an alkylating agent is present in an extracellular vesicle or an exosome.

In certain embodiments, a chemotherapy drug comprises an anthracycline. In certain embodiments, an anthracycline comprises one or more of the following drugs: daunorubicin, doxorubicin, doxorubicin liposomal, epirubicin, idarubicin, mitoxantrone, or valrubicin. In certain embodiments, an anthracycline is present in an extracellular vesicle or an exosome.

In another embodiment, a chemotherapy drug is an antitumor antibiotic. In certain embodiments said antibiotic comprises one or more of the following compounds: mitomycin C, dactinomycin, actinomycin D, bleomycin, or daunomycin. In certain embodiments, an antibiotic is present in an extracellular vesicle or an exosome.

In another embodiment, a chemotherapy drug comprises one or more of the following compounds: hydroxyurea, tretinoin, arsenic trioxide, a proteasome inhibitor (e.g., bortezomib), asparaginase, eribulin, ixabepilone, mitotane, omacetaxine, pegaspargase, procarbazine, romidepsin, or vorinostat. In certain embodiments, one or more of said drugs are present in an extracellular vesicle.

In certain embodiments, a chemotherapy drug is delivered within a nanoparticle that is configured to encapsulate and deliver a chemotherapeutic agent to a subject diagnosed with cancer. A nanoparticle may comprise one or more of the following: a liposome, an exosome, a protein-based nanoparticle, a transferrin-link nanoparticle, a folate-linked nanoparticle, a hyaluronic acid-linked nanoparticle, and an albumin-based nanoparticle. A chemotherapy drug may comprise a nanoparticle injectable suspension of paclitaxel, marketed as Abraxane®, comprising an albumin-bound form of paclitaxel with a mean particle size of approximately 130 nm.

In certain embodiments, a chemotherapy drug that is a target of the devices and methods disclosed herein comprises an antibody-drug conjugate (or an “ADC”) or a cytotoxic drug portion thereof. An ADC is typically comprised of of an antigen-specific monoclonal antibody, a cytotoxic drug, and a linker that conjugates the cytotoxic drug to the monoclonal antibody, however, it is appreciated that other molecules, moieties, or compounds may be added. ADC comprise two main classes of linkers: cleavable and non-cleavable. Cleavable linkers contain chemical or enzymatic liable chemistries that are formulated for rapid cleavage and release of the cytotoxic drug at the target site. Linkers are based on chemical motifs including but not limited to disulfides, hydrazones or peptides, or thioethers. In certain embodiments, an ADC comprises of one of the following drugs: Gemtuzumab ozogamicin, Brentuximab vedotin, Trastuzumab emtasine, Inotuzumab ozogamicin, Polatuzumab vedotin, Enfortumab vedotin, Trastuzumab deruxtecan, Sacituzumab govitecan, Belantamab mafodotin, Trastuzumab duocarmazine, Mirvetuximab soravtansine, Tusamitamab ravtansine (SAR408701), Inotuzumab ozogamicin, Moxetumomab pasudotox, Tisotumab vedotin, or Loncastuximab tesirine. In some embodiments, the cytotoxic drug is a small molecule, a cellular toxin, a protein toxin, a protein, an enyme, a radionuclide, or portions of fragments thereof.

In certain embodiments, an ADC comprises a cytotoxic drug comprising one or more of the following compounds, molecules, metabolites, or derivatives thereof: calicheamycins, auristatin derivatives (monomethyl auristatin E and monomethyl auristatin F), active metabolite of the topoisomerase I inhibitor irinotecan (SN-38), exatecan derivative (DXd), maytansinoid, derivatives of maytansine (DM1, DM4), duocarmycins, camptothecin derivatives, maytansinoid, pyrrolobenzodiazepine (PBDS), and the 38 kD fragment of Pseudomonas exotoxin A (PE38). In other embodiments, an ADC comprises a cytotoxic drug or compound comprising: a carmaphycin, an RNA inhibitor, a Bcl-xL inhibitor, or a niacinamide phosphate ribose transferase (NAMPT) inhibitor. In certain embodiments, an ADC comprises one or a plurality of cytotoxic drugs or compounds.

Theoretically, ADCs were developed to enhance the selectivity of chemotherapy drugs by targeting a cytotoxic drug to a desired cell population using an antibody. However, there are limitations in the safety of ADC that stem from unstable linkers such that the cytotoxic drug deconjugates from the monoclonal antibody and falls off in circulation. By some estimates, only ˜0.1% of the injected dose of an ADC is delivered to the tumor site. It is contemplated that aside from physiological drug clearance mechanisms, a substantial fraction of total drug could theoretically be present in circulation. Off-target delivery of circulating drug into healthy cells is also believed to be efficient, particularly for certain lipophilic cytotoxic drugs that exhibit high permeability. Many of the novel and highly cytotoxic drug components of ADC show significantly more toxicity (100-1000× more) than conventional chemotherapy drugs and confer significant adverse events. In some embodiments, extracorporeal systems and methods are provided for improving the safety and efficacy of an ADC by adsorbing and removing an ADC or a portion thereof. In particular, the devices and methods of the invention are useful for clearing the deconjugated or free cytotoxic drug component of an ADC from blood or plasma.

Chemotherapy drugs are typically administered to a subject intravenously or orally or by one or more other routes including but not limited to via injection subcutaneously, intraperitoneally, intramuscularly, intradermally, intraorbitally, intracapsularly, intraspinally, intrasternally, or the like, including infusion pump delivery, or administration locally, e.g., by depot implantation; or topically.

As used herein, the term “metabolite” refers to a byproduct of metabolism of the chemotherapy drug. Of relevance as targets of the devices and methods of the invention are drug metabolites that exert toxicity against healthy tissues. Non-limiting examples of drug metabolites include doxorubicinol (metabolite of doxorubicin and associated with cardiotoxicity) and 4-hydroxycyclophosphamide and acrolein (active, toxic metabolites of cyclophosphamide). In certain embodiments, the devices and methods of the invention are applied to reduce or eliminate or deplete a metabolite of a chemotherapy drug from blood or a blood component.

As used herein, the term “liposome” refers to a bilayer sphere of lipids, typically between approximately 20 nm and 1000 nm in diameter. A liposome may have similar physical properties as an exosome, for example, a similar lipid bilayer composition and/or a similar size. In certain embodiments, a liposome may comprise one or more of the following lipids: cholesterol, phosphatidylcholine, hydrogenated soy phosphatidylcholine, dipalmitoyl phosphatidylcholine, phosphatidylglycerol, phosphatidic acid, phosphatidylethanolamine, 2-distearyl-sn-glycero-3-phosphoethanolamine, phosphatidylserine, N-[1-(2,3-dioleyloxy)propyl]-N,N,N-triethylammonium (DOTMA), and 1,2-dioleoyl-3-trimethylammoniopropane (DOTAP). In certain embodiments, a liposome may be used to encapsulate a drug (i.e., a liposomal drug formulation) for the purpose of modifying the pharmacokinetic and/or pharmacodynamic properties of said drug, for example, by enhancing drug solubility, by protecting against drug degradation, and/or by enhancing the circulating half-life of a drug.

In some embodiments, a liposomal drug formulation comprises a mixture of encapsulated and non-encapsulated fractions of a chemotherapy drug. Loading of drugs into liposomes can be performed using methods known to one of skill in the art, for example, using thin lipid film hydration, reverse phase evaporation, microfluidic techniques, or dehydration-rehydration methods. In certain embodiments, a liposome has a molecule, compound, or ligand that is functionalized to its surface. In certain embodiments, a liposome is functionalized with polyethylene glycols (PEG), an aptamer, an antibody, a protein, a peptide, a ligand, a carbohydrate, or a small molecule. In certain embodiments, a liposomal drug formation is Doxil®, comprising the chemotherapy drug doxorubicin in a PEGylated liposomal formulation. Since a liposomal drug commonly has a longer half life in plasma as compared to a free, non-liposomal formulation of the same drug, it is contemplated that a need may arise to sequester or remove the excess or remaining liposomal drug in a patient to whom said drug had been administered. A liposomal drug formulation may lack specificity for the tumor, therefore, an undesired effect may be distribution of drug to non-target tissues.

The devices and methods described herein are useful for the removal of liposomes from blood or a blood component of a subject. In certain embodiments, a liposomal drug formulation or a portion thereof binds to an adsorbent in an extracorporeal device of the present invention. In preferred embodiments, said liposome drug formulation comprises one or more of the following drugs: DaunoXome® (comprising the chemotherapy drug daunorubicin), DepoCyt® (cytarabine), Doxil® (doxorubicin), Lipo-Dox® (doxorubicin), Marqibo® (vincristine), Mepact® (mifamurtide), Myocet® (doxorubicin), Onivyde®/Nal-IRI (irinotecan), Vyxeos®/CPX-351 (cytarabine: daunorubicin), Zolsketil® (doxorubicin), L-NDDP/Aroplatin™ (cis-bis-neodecanoato-trans-R,R-1,2-diaminocyclohexane platinum [II]), Endo-TAG® (paclitaxel), PLM60 (mitoxantrone), ThermoDox® (doxorubicin), LiPlaCis (cisplatin), Lipoplatin™ (cisplatin), or SPI-077 (cisplatin). In other embodiments, a liposomal drug formulation is designed or modified for targeted chemotherapy drug delivery to a tumor site, wherein said liposomal drug formulation also comprises an aptamer, an antibody, a Fab fragment of an antibody, or a portion thereof. Non-limiting examples of components of a liposomal drug formulation may include monoclonal antibodies or fragments or portions thereof that are directed against human epidermal growth factor receptor 2 (HER2), epidermal growth factor receptor (EGFR), epithelial cell adhesion molecule (EPCAM), CD19 (a B cell receptor), or vascular receptors on tumor endothelial cells. In certain embodiments, the systems and methods disclosed herein are utilized to bind to the liposome component of a drug that also comprises an aptamer, an antibody, a Fab fragment of an antibody, or fragments or portions thereof has been conjugated.

As used herein, “efficacy” of a treatment in reference to a drug or device therapy refers to the observation of a desirable tumor response for one or more relevant clinical endpoints. Response evaluation criteria in solid tumors or RECIST (version 1.1) refers to a set of published rules used to assess tumor burden for providing an objective assessment of response to therapy across specific tumor types and clinical trial settings. RECIST is used to classify tumor response categories of complete response (CR), partial response (PR), stable disease (SD), or disease progression. RECIST defines progressive disease as at least a 20% increase in the sum of diameters of up to 5 target lesions (2 lesions/organ), taking as reference the smallest sum on study and an absolute lesion increase of at least 5 mm or the appearance of new lesions. CR is defined the disappearance of all target lesions, and a PR is defined as at least a 30% decrease in the sum of the target lesions. SD is defined as fitting the criteria neither for progressive disease nor a partial response. In clinical trials, categorical responses for all patients are summated into radiologic image-based outcome measures including objective response rate (ORR), progression-free survival (PFS), overall survival (OS), and duration of survival. OS measures how long a subject survives on a treatment regimen. PFS defines the survival duration of a subject without disease worsening (i.e., a measure of disease control and stabilization). ORR is the proportion of subjects on a treatment regimen whose experience a complete response or a partial response. Duration of response is the length of time that a tumor continues to respond to treatment with tumor progression.

As used herein, an adverse event is any unfavorable and unintended side effect, symptom or disease resulting from a therapeutic intervention such as a drug or a device.

As used herein, “CTCAE” refers to Common Terminology Criteria for Adverse Events is the National Cancer Institute's widely accepted standard classification and severity grading scale for adverse events associated with cancer therapies. In CTCAE, an adverse event includes any abnormal clinical finding temporally associated with the use of a therapy for cancer. These criteria are used for identifying toxicities and establishing dosing for cancer therapies. The grading criteria for adverse events using CTCAE are as follows:
Grade 1: asymptomatic or mild symptoms, no intervention needed;
Grade 2: moderate symptoms, minimal, local or non-invasive intervention;
Grade 3: severe or medically significant symptoms but not immediately life threatening, hospitalization or prolongation of hospitalization indicated, disabling and limiting self care;
Grade 4: Life threatening consequences, urgent intervention required;
Grade 5: Death related to adverse event(s)

As used herein, “dose-limiting toxicities” (DLTs) are defined as severe toxicities or adverse events that occur in a subject during the first cycle or early phases of systemic cancer therapy such as chemotherapy. Toxicities are determined by the individual treatment protocol and using the CTCAE classification, wherein DLTs may be defined as all grade 3 or higher toxicities with certain exceptions; for example, grade 3 nonfebrile neutropenia and alopecia or other complications such as nausea and vomiting that can be controlled with the appropriate supportive care. In some circumstances, DTLs may be defined as irreversible grade 2 toxicities (e.g., neurotoxicities, ocular toxicities, or cardiac toxicities) or prolonged grade 2 toxicities. Acceptable DLTs may vary with the anticipated outcome of the treatment, for example more severe adverse events may be deemed accepted for a treatment regimen that is anticipated to be curative.

As used herein, the term “maximum tolerated dose” (MTD) refers to the highest dose of a specific drug that can be administered to a subject with an acceptable level of toxicity. In clinical oncology, dose escalation studies are normally performed to identify the MTD.

As used herein, the terms “formulation”, “drug formulation” and “composition” are used interchangeably to refer to a composition of matter for administration to a subject.

As used herein, “fluid” may refer to a biological fluid such as blood or a blood component (such as plasma), that may contain one or more of cells, antibodies, cytokines, peptides, proteins, molecules, or vesicles (e.g., extracellular vesicles), wherein one or more of these components is a target of an extracorporeal device disclosed herein. A fluid may also refer to a solvent, buffer, or other solution that is utilized in other aspects of manufacturing, testing, or priming the extracorporeal devices disclosed herein.

As used herein, the term “blood component” refers to a portion of whole blood (e.g., a fluid or cellular component) from a subject, wherein the whole blood has been processed or separated, for example, by centrifugation. In certain embodiments, blood into separated into one or more components, for example, into a white blood cell component and a plasma component.

As used herein, the term “plasma component” refers to a portion of plasma from a subject, wherein said portion may comprise a fraction of total plasma by volume or a portion of plasma constituents, for example, a portion of molecules, vesicles, proteins, mineral salts, sugars, fats, hormones, vitamins, or non-physiologic compounds, which may be physically separated based on size, charge, affinity to a substrate, or other physical or biochemical characteristics.

As used herein, the term “extracorporeal device” or refers to a cartridge or a column through which a fluid passes, preferably blood or a blood component, which comprises an internal compartment that contains material that interacts with the fluid. An extracorporeal device may be of various configurations with respect to size and shape, and may comprise one or more substrates, adsorbents, and/or ligands. In certain embodiments, an extracorporeal device is a commercial plasmapheresis device. Non-limiting examples including Plasmart plasma filters from Medica SPA (Modena, Italy) comprising hollow fiber polyethersulfone membranes.

As used herein, the term “target” refers to an entity in blood or a blood component that is subject to or is desired for removal by an extracorporeal device of the invention. For example, a target may comprise a chemotherapy drug, a metabolite of a chemotherapy drug, an extracellular vesicle such as an exosome, a molecule or drug that is associated with or contained within an extracellular vesicle, or portions thereof. In the context of the invention, a target may also comprise a nanovesicle, which may broadly refer to any liposomal drug or other nanocarrier-based drug as well as an exosome that may be naturally occurring. In embodiments that reference a “target comprising a chemotherapy drug”, this may refer to the drug compound itself but may also include an extracellular vesicle or an exosome that contains or is associated with a chemotherapy drug.

As used herein, the term “substrate” refers to a material that provides structure and a surface area in an extracellular device and to which a target factor can be either directly or indirectly bound. A substrate may comprise a membrane or resin for which a target factor has an affinity or attraction. In some embodiments, a substrate may comprise an adsorbent and, in the context of the invention, these terms may be used interchangeably. In other embodiments, a substrate may not directly provide an adsorptive surface. A substrate may comprise a membrane, surface, or material to which one or more affinity capture molecules or ligands (e.g., an aptamer, an antibody, a Fab fragment, a small molecule, or fragments thereof) are conjugated or attached for binding to a target factor.

As used herein, the term “adsorbent” refers to a substrate, medium, ligand, molecule or compound that adsorbs or binds to a factor, for example, a molecule, a moiety, a compound, a drug, or an extracellular vesicle. Typically, adsorption involves binding or holding of a target factor by chemical attraction to a surface. Non-limiting examples of adsorbents include activated carbon, ion exchange resins, and non-ionic exchange resins. An adsorbent may possess one or more properties that dictate its binding to a factor, including but not limited to its functional groups (e.g., base vs. acid vs. non-ionic), its porosity (e.g., macroporous vs. gel), and its matrix composition (e.g., polystyrene, polyacrylic etc.). An adsorbent may have properties that facilitate binding or retention of one or more factors, e.g., a bead composition and size that permits sequestration of one or more factors in the matrix. An adsorbent may also be subjected to chemical modifications to endow additional binding or adsorptive features, for example, by conjugating a ligand to the adsorbent. As used herein, an “adsorbent component” refers to one or more adsorbents used for binding one or more targets from a fluid or a blood component.

As used herein, the term “extracellular vesicle” broadly defines a cell-derived membrane vesicle that can be separated from non-membranous particles in blood or a blood component. Extracellular vesicles include microvesicles, exosomes, oncosomes, and apoptotic bodies, or other vesicles that are secreted or pinched off the surface of parent cells. Extracellular vesicles are variable sizes but generally range from approximately 30 nm up to 1,000 nm in diameter. In certain embodiments, an extracellular vesicle comprises an antigen, an epitope, a molecule, a lipid, a protein, a glycoprotein, a glycolipid, on its surface that binds to an adsorbent.

As used herein, the term “exosome” refers to a bi-lipid membrane vesicle of approximately 30-150 nm in diameter (average size of approximately 100 nm) that is secreted by a cell. In the context of the invention, an exosome is a type of extracellular vesicle. Exosomes may carry components from cell membranes or from a cell's components including proteins and nucleic acids including but not limited to messenger RNA (mRNA), microRNA (miRNA), long-non-coding RNA (IncRNA), and circular RNA (circRNA) that can be transmitted to recipient cells. In certain embodiments, exosomes are identified by the expression of surface proteins unique to the endosomal pathway or fragments thereof including but not limited to tetraspanins such as CD9, CD63, CD81, heat shock proteins (e.g., HSP70), lysosomal proteins (e.g., Lamp2b), and the tumor-sensitive gene 101 (Tsg101), and fusion proteins (e.g., flotillin and annexin). It is known in the art that exosomes are present in higher concentrations in blood or a blood component in a subject with a disease state such as cancer as compared to their concentrations in the same subject prior to disease, during disease stabilization with partial/full resolution of disease, or during abatement of symptoms, or in comparison to another subject who is disease-free. It is also known in the art that exosomes are released in abundance by diseased cells (e.g., tumor cells), and contain cargo from diseased cells, which is then released into the systemic circulation where uptake of said exosomes by other cells may occur, including non-diseased cells and other tumor cells. It is established in the art that a tumor's response to a chemotherapy drug involves increased exosome production, wherein the drug-exposed tumor actively pumps out exosomes containing the drug along with its other disease promoting cargo.

As used herein, the term “nanovesicle” refers to any nano-sized vesicle that may be naturally occurring (e.g., exosomes), synthetic (e.g., liposomes, ethosomes), or bioengineered hybrid vesicles. Nanovesicles are characterized by a membranous lipid bilayer that comprises phospholipids. Typically, all nanovesicles fall in the 50-500 nm size range but may be smaller (<200 nm for exosomes and liposomal drug formulations).

As used herein, the term “protein” refers to a macromolecule comprised of amino acids linked by peptide bonds. Proteins and fragments or portions thereof (i.e., peptides) are manufactured biologically inside of a cell or can be produced synthetically in a laboratory. In the context of the invention, peptides, proteins, or fragments of portions thereof may be packaged inside an extracellular vesicle or an exosome, or they may be displayed on the surface of said extracellular vesicle or exosome.

As used herein, the term “nucleic acid” refers to a molecule comprising deoxyribonucleic acid (DNA), ribonucleic acid (RNA), an oligonucleotide, a modified nucleotide (e.g., having modified sugars or bases), fragments thereof, monomers thereof, analogs thereof, or combinations thereof. Nucleic acids can be either single stranded or double stranded. In certain embodiments, a nucleic acid comprises non-coding RNA (ncRNA), which may comprise short-chain (<200 nucleotides) non-coding RNA such as microRNA (miRNA), long-non-coding RNA (IncRNA; >200 nucleotides), and circular RNA (circRNA). The functions of these nucleic acids are to modify transcription of particular genes. Certain non-coding RNAs have a role in the chemotherapy resistance of tumors as well as other aspects of malignancy. In some embodiments, non-coding RNA is contained with an extracellular vesicle or an exosome.

As used herein, the term “aptamer” refers to a short, single-stranded DNA or RNA (ssDNA or ssRNA) molecule that can selectively bind to a specific target, for example, a protein, a peptide, a carbohydrate, a toxin, or a nanovesicle. As used herein, “aptamer” is also used to encompass a modified aptamer such as a slow off-rate modified aptamers (SOMAmers), which have additional side chains that enhance aptamer stability.

As used herein, the term “lectin” refers to a carbohydrate-binding protein found in or derived from living organisms that bind to carbohydrate structures (i.e., glycans). Lectins possess distinct specificity for different glycan motifs. For example, a certain lectin or group of lectins may bind to a particular surface glycan present on a cell membrane. Specific lectins also exhibit binding to the membrane surfaces of extracellular vesicles such as exosomes and, therefore, are considered useful affinity agents for binding extracellular vesicles present in a fluid, for example, using an extracorporeal device.

### Methods of Treatment or Use

The invention provides systems and methods that are useful for improving the activity of a chemotherapy drug against tumors by addressing circulating drug resistance factors and by removing factors that interfere with a drug's safety and efficacy. The invention satisfies the need for methods of targeting entities from the circulatory system that underlie chemotherapy treatment challenges, wherein the entities preferably comprise exosomes and chemotherapy drug. For achieving these objectives, the methods of the invention provide extracorporeal devices comprising adsorbents, wherein the adsorbents preferably comprise activated carbon and/or least one non-ionic exchange resin. In certain embodiments, the extracorporeal methods of the invention provide adsorbents comprising activated carbon, a non-ionic aliphatic ester resin and a non-ionic polystyrene divinyl benzene resin. In other embodiments, the extracorporeal methods apply adsorbents comprising an ion exchange resin. In certain embodiments, an adsorbent component in an extracorporeal device may comprise ion exchange resin combined with one or more additional adsorbents comprising activated carbon or non-ionic exchange resins.

The methods of the present invention can be practiced using one or a plurality of extracorporeal blood purification devices having either identical or different physical structures and adsorbent compositions. In a preferred embodiment, the methods include two extracorporeal blood purification devices for treating a subject with cancer. In certain embodiments, a first extracorporeal device is used for clearing targets from blood or plasma from a subject prior to administration of a chemotherapy drug, wherein the targets of interest are induced or produced by a tumor or by other disease-affected cells in the subject. In preferred embodiments, the targets comprise exosomes that have deleterious roles involving interference with the activity of the chemotherapy drug, mediating drug resistance, and spreading malignant cargo including proteins and nucleic acids from a tumor and/or from disease-affected cells. In certain embodiments, a chemotherapy drug is then administered to the subject and, following tissue distribution of the drug, an extracorporeal method is then performed using a second device for the purpose of clearing or reducing deleterious targets from blood or plasma that result from or are incited by said drug administration. In certain embodiments, the second device removes a fraction of blood or plasma that comprises a chemotherapy drug. In other embodiments, the second device removes exosomes that are carriers of chemotherapy drug and other tumor resistance factors. Preferred methods of the invention provide for the simultaneous removal of exosomes and chemotherapy drug by the second device. In other preferred embodiments, the disclosed methods provide for the simultaneous removal of exosomes, chemotherapy drug, and metabolites of said chemotherapy drug by the second device, wherein the metabolites are by-products of drug metabolism but exert cytotoxic activity and mediate adverse events in a subject. Methods using the second device in the post-chemotherapy period can be performed to reduce treatment toxicity by reducing the bloodstream presence of chemotherapeutic drug agents that are not delivered to the tumor site. In some embodiments, methods using the second device are applied to reduce the bloodstream presence of chemotherapy-induced targets such as exosomes that promote the spread of cancer metastasis. In other embodiments, the extracorporeal methods using a first device are performed to augment the benefits of treatment with a second device by facilitating increased removal of exosomes that may compete for target site binding or otherwise adversely impact the performance of a second device for reducing toxic drug levels.

In certain embodiments, the invention provides a method of improving the safety and efficacy of a chemotherapy drug in a subject comprising the following steps: (a) providing a first extracorporeal device comprising an adsorbent component; (b) connecting said device to an extracorporeal system; (c) passing blood or plasma comprising exosomes through said extracorporeal device, wherein the adsorbent component reduces the presence of exosomes in said blood or plasma; (d) administering a chemotherapy drug to the subject; and (e) introducing blood or plasma comprising exosomes and chemotherapy drug through a second extracorporeal device comprising an adsorbent component, wherein the adsorbent component reduces the presence of exosomes and chemotherapy drug in said blood or plasma. Optionally, the exosomes are identified or quantified in a blood sample from said subject prior to (c) or after (c) or both, and/or prior to (e) or after (e) or both. In some embodiments, one or a plurality of adsorbents are applied in steps (c) and (e). In certain embodiments, it is advantageous to provide a plurality of adsorbents to capture and remove targets with different sizes and/or to provide additional biochemical mechanisms of action for adsorption of targets of interest. Certain embodiments provide extracorporeal methods that use the same adsorbent components for treating a subject prior to (pre-chemotherapy) and following administration of a dose of drug (post-chemotherapy). In certain embodiments, the methods include treatment of a subject with either the first or second device, depending on the clinical needs of the subject. Beneficial or desirable clinical results from applying the disclosed extracorporeal methods may include amelioration or diminishment of adverse events resulting from the drug, improved tolerability of the drug therapy, improved treatment compliance with the drug regimen, and improved tumor responses to therapy.

Specific embodiments provide methods for eliminating or reducing the levels of a chemotherapy drug in blood or plasma of a subject comprising: (a) identifying a subject who has received at least one dose of a chemotherapy drug, which in certain embodiments, may comprise a liposomal drug formulation; (b) introducing blood or plasma comprising free drug and drug-containing exosomes into an extracorporeal device, wherein said extracorporeal device contains an adsorbent comprising activated carbon and one or more non-ionic exchange resins; (c) contacting the blood or plasma with the adsorbent for a time sufficient to allow free drug and exosomes present in blood or plasma to bind to the adsorbent; (d) reintroducing the blood or plasma obtained after (c) into the subject, wherein the blood or plasma obtained after (c) has a reduced amount of free drug and exosomes, as compared to blood or plasma prior to b); (e) optionally, identifying and quantifying the drug and/or the exosomes present in a plasma sample from the subject prior to (b) and/or after (c) or both. Quantitative measurements of the targets can be used to inform whether treatment with the disclosed methods should be continued or repeated to reduce total chemotherapy drug levels to the desired levels. For example, the extracorporeal methods may be performed twice daily, at least once daily for 1-7 days, or at less frequent intervals following drug administration. Additionally, in certain embodiments, one or more additional extracorporeal procedure may be performed prior to administration of the chemotherapy drug to the subject for the purpose of pre-clearing exosomes from circulation that potentially interfere with the efficacy of the post-chemotherapy drug removal device.

Aspects of the invention involve quantifying a reduced amount of a chemotherapy drug following the performance of an extracorporeal procedure, wherein a reduced amount in the blood or plasma is deduced by comparison to a measurement of the drug levels prior to the procedure. In some embodiments involving quantification of a chemotherapy drug, a patient to whom a chemotherapy drug has been administered is selected, a blood specimen is obtained from said subject, and an established methodology in the art is applied to the specimen for measuring the levels of said drug, which may comprise one or more of liquid chromatography mass spectrometry (LC-MS), high performance liquid chromatography (HPLC), ultra high performance liquid chromatography (UHPLC), chemiluminescence, electroluminescence, surface plasmon resonance (SPR), enzyme linked immunosorbent assay (ELISA), and/or other techniques.

Embodiments of the invention involve selecting a subject for an extracorporeal method disclosed herein for whom treatment with a chemotherapy drug is planned. Alternatively, a subject who has received one or more doses of a chemotherapy drug as part of a treatment regimen may be selected. In certain embodiments, a patient who has experienced chemotherapy-associated adverse events resulting from one or more doses of a chemotherapy drug is selected for therapy with the disclosed methods. In other embodiments, a subject is selected who has failed treatment with at least one chemotherapy drug, wherein the subject is deemed to be a “non-responder” who did not respond to the chemotherapy treatment regimen, or the subject has developed progressive disease during treatment. In clinical oncology practice, chemotherapy treatment failure to a first treatment regimen often results in a change of drug(s) to a second treatment regimen, where the second treatment regimen comprises drugs that have a different mechanism of action. Alternatively, in cases where treatment options have been exhausted, treatment of the subject's cancer may be discontinued entirely. Embodiments of the invention include extracorporeal methods for preventing chemotherapy treatment resistance to one or more drugs comprising a treatment regimen for a subject. Other embodiments of the invention comprise extracorporeal methods for a subject who has experienced treatment failure to a first treatment regimen, wherein the methods disclosed herein are applied in conjunction with a rechallenge to one or more drugs in the first treatment regimen or with one or more drugs comprising a second treatment regimen.

Other embodiments of the invention involve selecting a subject for treatment with the disclosed methods by identifying, characterizing, or quantifying an exosome population in blood or plasma of the subject. In certain embodiments, implementation of the methods disclosed herein is informed by measurement of the total concentrations of exosomes in blood or plasma. Methods for isolating and quantifying exosomes are known to those of ordinary skill in the art and may involve steps of identifying exosomes based on their surface molecule expression or their cargo. Non-limiting examples for these methods include measuring the isolated exosomes/mL in plasma, wherein exosomes are identified by one or more molecules comprising tetraspanins such as CD9, CD63, CD81, heat shock proteins (e.g., HSP70), lysosomal proteins (e.g., Lamp2b), the tumor-sensitive gene 101 (Tsg101), and fusion proteins (e.g., flotillin and annexin). The steps of identifying and quantifying exosomes in a subject may be performed prior to and/or following administration of a chemotherapy drug, wherein an increase in endogenous exosomes in blood or plasma may serve as a biomarker of tumor activity and/or drug resistance.

Methods of the invention also include quantifying exosomes in a subject prior to and following the performance of an extracorporeal procedure and determining the percentage of total exosomes that were removed by a device disclosed herein. In certain embodiments, exosome concentrations in plasma are monitored to inform whether optimization, extended duration, or repetition of an extracorporeal procedure is required to reduce the exosome levels to a desired level. Beneficial outcomes of the extracorporeal methods involve observing a reduction in the quantities or concentrations of exosomes following treatment of a subject with the extracorporeal devices described herein. In some embodiments, methods of the invention comprise determining whether a subject has elevated levels of exosomes in blood or plasma compared to a control level, i.e., a level present in a disease-free individual, an individual not experiencing a disease symptom, or from the same subject at an earlier or pre-disease stage. By way of example, an elevated concentration of exosomes relative to a control may serve as an indicator for implementation of the methods disclosed herein or for an increased frequency of applying the methods disclosed herein.

Other methods of the invention comprise evaluating the composition of microRNAs (also referred to as miRNAs), specifically microRNAs with roles in resistance to certain chemotherapy drugs, in exosomes from blood or plasma of a subject. Non-limiting examples of microRNAs that are associated with chemotherapy resistance include miRNA-320a, miRNA-205, miRNA-7, miRNA-519c, miRNA-384, miRNA-96, miRNA-499a, miRNA-125a, miRNA-224, miRNA-27b-3p, miRNA-21, miRNA-134, miRNA-125b, miRNA-449, miRNA-508-p, miRNA-103/107, miRNA-495-3p, and others. Long non-coding RNAs (InRNAs) are similarly transported in exosomes and participate or are associated with drug resistance; non-limiting examples include IncRNA UCA1, IncRNA AX747207, IncRNA ROR, and others. Methods known in the art can be applied for isolating exosomes and quantifying microRNAs and IncRNAs using quantitative real-time PCR assays, miRNA microarrays or other techniques. Methods of the invention therefore comprise isolating exosomes from a sample of blood or plasma of a subject and quantifying the microRNA and/or IncRNA expression profiles in said exosomes. The quantities of one or more microRNAs in an exosome isolate can be compared to a control (i.e., a quantity present in a disease-free individual, an individual not experiencing a disease symptom, or from the same subject at an earlier or pre-disease stage.) Beneficial outcomes of the extracorporeal methods disclosed herein involve observing a reduction in presence and/or the quantities of one or more microRNAs and/or IncRNAs that are present in a volume of plasma and/or in an exosome isolate. Additionally, a need for applying the extracorporeal methods disclosed herein or a need for an increased frequency of performing said methods can be informed based on high or increased quantities of microRNAs and/or IcnRNAs in exosomes of a subject relative to an earlier time for the subject or relative to control subject(s) that are disease free or responsive to treatment.

Disclosed herein are extracorporeal methods that are applied in conjunction with different classes or categories of chemotherapy drugs. The phrase “in conjunction” as used herein can refer to application of an extracorporeal method prior to or following administration of a dose of drug or at both time points. Chemotherapy drugs are classified based on their mechanisms of action in the body, i.e., based on their effects at the cellular or molecular level in tumors. The extracorporeal methods of the present invention are directed toward ameliorating extracellular vesicles or exosomes, which represent a universal mechanism of diseased cells for spreading malignant cargo and drug resistance factors. Also disclosed herein are treatment methods that can be implemented at any time intervals during a treatment regimen with a chemotherapy drug (i.e., when multiple doses of drug are given on specific days over a time interval, usually spanning weeks or months). The invention also provides extracorporeal methods for use in therapeutic regimens involving a single chemotherapy drug, a plurality of chemotherapy drugs, or one or more chemotherapeutic drugs combined with other oncologic agent(s), for example, combined with targeted therapies, immune checkpoint inhibitors, hormone therapy, small molecule inhibitors, or other therapeutic molecules, compounds, and/or vaccines.

In some embodiments, the systems and methods disclosed herein are used in combination with molecules, drugs, or compounds that inhibit extracellular vesicle or exosome production or release by cells. Inhibitors of exosome production or release for use with the systems and methods disclosed herein comprise one or more of the following compounds or drugs: pantethine, imipramine, spiroepoxide, DPTIP [2,6-dimethoxy-4-(5-phenyl-4-thiophen-2-yl-1H-imidazole-2-yl)-phenol] GW4869, simvastatin, glibenclamide (glyburide), indomethacin, calpeptin, bisindolylmaleimide I, Y-27632, U0126, manumycin A, dimethyl amiloride, tipifarnib, ketoconazole, endothelin A receptor antagonist, cannabidiol, ketotifen, lansoprazole, omeprazole, esomeprazole, pantoprazole, dynasore, and methyl-beta-cyclodextrin. Each of these drugs or compounds can be administered at doses or dosing regimens as deemed to be effective by one of skill in the art. The extracorporeal devices disclosed in the present invention can be applied prior to and/or following the administration of a chemotherapy drug to a subject in conjunction with one or more exosome inhibitors. This embodiment of the invention provides methods for physical removal of exosomes from circulation using an extracorporeal device as well as the simultaneous reduction or amelioration of de novo generation or appearance into the circulatory system.

Disclosed herein are methods for removing or depleting chemotherapy-induced exosomes drug from blood or plasma following administration of a dose of said chemotherapy drug to a subject in need thereof. It is established in the art that tumor cells possess drug efflux pumps that actively remove chemotherapy drug via exosomes, wherein high concentrations of exosomes in circulation may be considered biomarkers of a tumor's drug resistance. One of skill in the art would determine the timing for applying the disclosed extracorporeal methods based on known drug pharmacokinetics and/or the anticipated onset of adverse events from published clinical trial results that are available for approved drugs. Advantageously, the methods are applied at an early stage following drug administration that adverse events are prevented or substantially reduced. Accordingly, certain embodiments of the invention involve measurement and quantification of chemotherapy drug found within exosomes from blood or plasma of a a subject to whom at least one dose of said chemotherapy drug has been administered. Methods for these assessments are known in the art but may include the use of techniques such as ultra-performance liquid chromatography to quantify the encapsulated chemotherapy drug in an isolated exosome fraction from blood or plasma of the subject.

Other embodiments of the invention involve quantifying total exosomes in blood or plasma to identify when an anticipated spike in exosome concentrations occurs following chemotherapy drug administration, whereby the total exosome quantities or concentrations in blood or plasma are compared prior to and following administration of the drug. The steps of identifying and quantifying exosome populations can be performed at various times post-drug administration for the purpose of discerning when an extracorporeal method of the invention should be performed; for example, after 12 hours, after 18, hours, after 24 hours, after 36 hours, after 48 hours, or longer after a dose of chemotherapy drug. Accordingly, disclosed herein in some embodiments are methods for removal of exosomes from blood or plasma of a subject comprising: (a) introducing blood or plasma from the subject into an extracorporeal device comprising an adsorbent component, wherein the adsorbent component comprises activated carbon or one or more non-ionic exchange resins or both; (b) contacting the blood or plasma from the subject with the adsorbent component in the device for a time sufficient to allow the exosomes to bind to the adsorbent component; (c) reintroducing the blood or plasma into the subject, wherein the blood or plasma has a reduced amount of exosomes after (b) as compared to the blood or plasma before (a). Optionally, these methods may be performed by detecting and/or quantifying the exosomes comprising chemotherapy drug and/or free circulating chemotherapy drug in blood or plasma of the subject.

The invention provides methods for reducing the incidence or severity of chemotherapy-associated adverse events that often lead to dose delays and modifications and therapy interruptions or discontinuation. Chemotherapy involves the disruption of cell replication or cell metabolism. Since chemotherapy generally has poor target specificity for tumor tissue, these drugs mediate significant off-target toxicity that is manifested as adverse events in treated patients. Low drug concentrations reach the tumor itself, e.g., only <1%, <2% or <5% for certain drugs. Non-limiting examples of adverse events resulting from a chemotherapy drug may include one or more of the following: nausea, vomiting, diarrhea, fatigue, constipation, anorexia, cytopenia (anemia, leukopenia, neutropenia, thrombocytopenia), mucositis, gastritis, peripheral neuropathy, hand-foot syndrome, type 1 hypersensitivity reactions, unusual bleeding or bruising, skin hyperpigmentation, skin necrosis, photosensitivity, neutrophilic eccrine hidradenitis, localized scleroderma, recall reactions, Raynaud's phenomena, vasculitis, thromboembolism, and susceptibility to infections. The adverse events are drug and dose-dependent with inter-patient variability in their appearance and severity. Chemotherapy-induced adverse events, if severe, often lead to hospitalization, or require treatment with analgesics or other medications to alleviate symptoms. In a clinical setting, the incidence and severity of adverse events can be informed using CTCAE grading criteria for adverse events in a subject who has already received at least one dose of a chemotherapy drug. By way of example, the disclosed methods may be useful for reducing the grade of an adverse event that is experienced a subject and/or to mitigate the onset of adverse events for a planned dosing of said subject with a chemotherapy drug.

The invention provides extracorporeal methods for overcoming dose-limiting toxicities (DLTs) of one or more chemotherapy drugs in a treatment regimen, wherein a clinically unacceptable toxicity of a drug or drug or drug combination is identified that prevents further administration of the drug(s) at the dose level at which said toxicity was observed. Additionally, methods are disclosed for increasing the maximum tolerated dose of said chemotherapy drugs, wherein this dose is the highest dose that is determined to have a clinically acceptable safety profile. In some embodiments, the extracorporeal methods described herein are utilized in conjunction with one or more chemotherapy drugs that are administered according to standard of care oncology practice, i.e., at the doses, schedule, cycle intervals, and duration of therapy (i.e., number of cycles) that has been clinically evaluated, approved by the appropriate regulatory agency, and/or that is prescribed by a medical practitioner in oncology. Drug doses are calculated for a subject in a conventional manner, for example on body weight, body surface area, flat dosing, and/or therapeutic drug monitoring for individualized dosing, where appropriate. For the standard or conventional dosing, the maximum tolerated doses for chemotherapy drugs are established in the prior art, normally from dose finding or dose escalation clinical studies, and are based on establishing the incidence of dose limiting toxicities in cohorts of subjects to whom escalating drug doses are administered. Desirable clinical results from applying the disclosed extracorporeal methods include an improved safety profile for the drug(s), which may be related to a reduced incidence or severity of adverse events, a shorter duration of adverse events, and an improved quality of life, for one or more treated subjects as compared to the same subject(s) prior to receiving the disclosed methods or one or more subjects receiving the identical chemotherapy treatment regimen without the disclosed methods. In some embodiments, the disclosed methods further comprise observing an increase in adherence of subjects to a chemotherapy treatment regimen, defined as fewer treatment interruptions or discontinuations and fewer dose modifications (i.e., dose lowering and/or shortening the treatment regimen. Further embodiments of the invention comprise administration of the disclosed extracorporeal methods and assessment of clinical endpoints of tumor responses using RECIST.

Other embodiments are contemplated wherein the extracorporeal methods of the invention are observed to lessen one or more dose limiting toxicities for a chemotherapy drug. A desired outcome of applying the methods of the invention to clinical practice includes the administration of a higher dose of one or more chemotherapy drug(s) to a subject without the adverse consequences of said drug(s), wherein the maximum tolerated dose for the drug(s) can be increased. In certain embodiments, the extracorporeal methods disclosed herein mitigate a dose limiting toxicity of one or more chemotherapy drugs, wherein a grade 3 or above toxicity at a drug dose level is modified to a grade 2 or lower toxicity in one or more subjects following implementation of the disclosed methods in conjunction with treatment with the chemotherapy drug(s). In other embodiments, the extracorporeal methods disclosed herein are suitable for increasing the maximum tolerated dose of a drug or a drug combination by at least 5%, at least 10%, at least 15%, at least 20%, at least 30%, at least 40% or at least 50% compared to a conventional or standard dose of said drug or drug combination. Since an established dose-response relationship exists between chemotherapy drugs and tumor regression, embodiments of the invention provide for improved clinical efficacy of one or more chemotherapy drugs. In other embodiments, it is contemplated that a subject's tolerance for a more aggressive chemotherapy treatment regimen, as afforded by the disclosed methods, may also provide earlier achievement of tumor responses in treated subjects. From a clinical standpoint, this advantage may translate into a reduction in the total dose of a drug given to the subject over a period of time, a reduction in the number of days of treatment, and/or a reduction in the number of treatment cycles required for achieving a clinical response in a subject.

The invention provides extracorporeal methods for preventing, reducing, or delaying the onset of chemotherapy drug resistance in a subject. Drug resistance can be classified as either intrinsic/primary resistance, wherein a disease is not initially responsiveness to exposure to one or more drugs, or it can be acquired resistance, wherein the disease ceases responding to one or more drugs that previously affected the disease process. In many embodiments, established biomarkers of drug resistance are lacking and resistance is instead inferred from clinical endpoints such as tumor response assessments using RECIST guidelines. Determination of drug resistance in a subject takes into consideration the known clinical response rates to the drug itself for the specific cancer type based. For example, tumor progression in a subject that previously had disease stabilization on a chemotherapy regimen may serve as an indicator that a tumor has become drug resistant. Once a subject has disease progression on a particular chemotherapy drug regimen, an accepted strategy is to initiate a different therapy (i.e., using a different drug/class of drug). Embodiments of the invention provide extracorporeal methods for treating a subject who is resistant to chemotherapy drug, wherein the methods of the invention allow the subject to be rechallenged with the same drug. In some embodiments, the disclosed systems and methods are applied for treating a subject that had progressive disease after initially responding to a first chemotherapy drug, wherein following a treatment-free time interval or upon treatment with a second drug, methods of the present invention are applied in conjunction with rechallenge with the first drug or treatment with the second drug.

The invention provides methods for addressing multidrug resistance (MDR), wherein a disease is resistant to more than one functionally and/or structurally unrelated drug. One form of multi-drug resistance is mediated by a membrane bound 170-180 kD energy-dependent drug efflux pump designated as P-glycoprotein (P-gp), a mechanism used by a tumor cell to “detoxify” itself by active removal of said drug from the cell. Non-limiting examples of drugs that are removed from tumor cells via P-glycoprotein include vinca alkaloids (vincristine and vinblastine), anthracyclines (e.g., doxorubucin, epirubicin, valrubicin, idarubicin, daunorubicin, mitoxantrone), topoisomerase II inhibitors (e.g., etoposide), and taxanes (e.g., paclitaxel, docetaxel, and cabazitaxel). The invention contemplates the use of the devices disclosed herein to lessen or mitigate the influence of exosomes that comprise P-glycoprotein, wherein said exosomes are released from a chemo-resistant tumor cell and transfer p-glycoprotein to a chemo-sensitive tumor cell. The invention thereby provides extracorporeal devices to mitigate the spread of chemotherapy resistance factors during the treatment regimen, including embodiments wherein said resistance factors are depleted prior to administration of a dose of a chemotherapy drug, following the administration of a chemotherapy drug or at both time points relative to drug dosing. In certain aspects of the invention, treatment with the extracorporeal devices disclosed herein is informed by measurement of P-glycoprotein in a blood component (e.g., in plasma or serum) of a subject prior to initiating said treatment. Methods for evaluating P-glycoprotein will be known to one of skill in the art and may involve steps of isolating and/or purifying exosomes from a blood component of said subject and utilizing the isolated material for a P-glycoprotein detection assay, for example, by Western blotting, flow cytometry proteomic analysis, or other known methods for assessing extracellular vesicle content. Other drug efflux pumps may be similarly evaluated instead or in addition to P-glycoprotein including but not limited to multidrug-resistant protein-1 (MRP-1) and breast cancer resistance protein (BCRP/ABCG2). In certain embodiments, the presence of one or more drug efflux pumps in an exosome from a blood or plasma of a subject serves as a biomarker of exosome-mediated acquisition and spread of drug resistance in the subject. The present invention thereby contemplates a treatment strategy for drug resistance, wherein said drug resistance is treated by removal or reduction of exosomes from blood or a blood component and wherein this treatment step may be performed prior to and/or following administration of a dose of a chemotherapy drug or both.

The invention provides extracorporeal devices that are suitable for achieving efficient or substantial reduction of high concentrations of disease-related exosomes in blood or plasma of a subject with cancer. In general, patients with cancer have higher concentrations of exosomes measurable in plasma or serum than disease-free individuals, which may be orders of magnitude higher in certain instances. Typically, patients with earlier stages of cancer have lower concentrations of exosomes than patients with advanced disease stages and higher tumor burdens. Methods for identifying and quantifying exosomes from a blood specimen are known in the art and include techniques such size exclusion chromatography, precipitation, ultracentrifugation, density gradient centrifugation, immunoaffinity methods and various biofluidics devices for exosome isolation followed by application of distinct methodologies such as nanoparticle tracking analysis for enumerating the exosomes. In some embodiments, a subject with cancer is identified and isolation and quantification of exosomes is performed prior to treatment with the devices disclosed herein. In certain embodiments, a subject that is selected for treatment with the disclosed methods has an exosome concentration measured in plasma or serum of at least 10⁸ exosomes/mL, 10⁹ exosomes/mL, 10¹⁰ exosomes/mL, 10¹¹ exosomes/mL or 10¹² exosomes/mL. Preferred extracorporeal devices and methods of the invention achieve the removal of a substantial fraction of exosomes present in blood or plasma of a subject, which in certain embodiments involves the removal of at least 10%, 15%, 20%, 25%, 30%, 40%, 50%, 60%, 70%, 80%, 90%, 95% or more of the exosomes present in blood or plasma of a subject.

Additionally, the methods described herein provide a first extracorporeal device for reducing the concentrations or levels of exosomes present in blood or plasma of a subject, wherein the first device may be utilized pre-chemotherapy drug dosing. The methods subsequently provide a second extracorporeal device for treating a subject post chemotherapy administration, wherein the second device simultaneously removes drug and exosomes that are continually being produced by diseased cells and that are induced following drug administration. It is also contemplated that treatment with a first device for treating a subject prior to chemotherapy can enhance the performance of the second device in terms of the clearance of toxic chemotherapy drug and chemotherapy-induced exosomes from blood or plasma. In some embodiments, the pre-clearing step using the first device is applied to increase the quantities of chemotherapy drug and exosomes that are removed by the second device. In certain embodiments, the performance of the second device may be evaluated by measuring the concentrations of exosomes present in a blood component such as serum or plasma of a subject after treatment with the first device but prior to treatment with the second device, in comparison to the concentrations of exosomes present following treatment with the second device.

In certain embodiments, the concentrations of a chemotherapy drug are also quantified in serum or plasma after treatment with the first device but prior to treatment with the second device. These measurements can be used to establish the fraction or percentage of each target that is removed from blood or plasma by the device. In certain embodiments, the second device achieves removal of at least 10%, 15%, 20%, 25%, 30%, 40%, 50%, 60%, 70%, 80%, 90%, 95% or more of the exosomes and/or the chemotherapy drug that are present in blood or plasma of a subject prior to the second procedure.

### Exemplary Extracorporeal Devices

Disclosed herein are extracorporeal devices for removal or depletion of chemotherapy-related targets in blood that compromise safety and efficacy of chemotherapy drugs. In specific embodiments, an extracorporeal device is configured to bind and capture a chemotherapy drug, a metabolite of a chemotherapy drug, an extracellular vesicle such as an exosome, a molecule or drug that is associated with or contained within an extracellular vesicle, or portions thereof.

In certain embodiments, an extracorporeal device is compatible for use with one or more commercially available, industry standard blood processing systems for performing hemodialysis, apheresis, continuous renal replacement therapy (CCRT), and therapeutic plasma exchange (TPE), to filter or remove one or more target factors from blood or a blood component. The devices described herein can be connected using blood tubing sets, thus allowing for devices to be configured for use with blood processing systems. Non-limiting examples of blood processing systems are offered by the following manufacturers: Dialco Medical, Baxter Healthcare, Haemonetics Corporation, Gambro, NxStage, Fresenius Kabi, Fresenius Medical Corporation, Asahi Kasei Kuraray Medical Co, Terumo BCT, Inc., HemaCare Corporation, Therakos, Inc, B. Braun Melsungen AG or Medica SPA.

Treatment of a subject with an extracorporeal device is initiated through access to a patient's circulatory system by connecting the extracorporeal lines of the catheter to the subject's circulatory system. In one embodiment, access is obtained through the insertion of a central venous catheter into a patient. In a preferred embodiment, the catheter is a dual lumen catheter. Prior to initiation of the treatment session, a primary solution, which may include a saline or albumin solution is advantageously circulated throughout the device to improve hemocompatibility. Optionally, an anticoagulant may be administered to the subject. Exemplary anticoagulants include but are not limited to unfractioned heparin, low-molecular weight heparin, citrate, and thrombin inhibitors. In certain embodiments, the extracorporeal circuit is “primed”, which refers to the process of flushing the system with one or more biologically compatible fluids prior to use. Priming may be performed using albumin to improve the biological compatibility and/or to quench the substrate in the extracorporeal device to decrease off-target binding of plasma proteins. Once the device has been primed and access to the circulatory system has been established, a pump is used to facilitate flow of blood or a blood component through the extracorporeal device. The pump can be any approved device suitable for facilitating extracorporeal filtration of blood and/or plasma. As the subject's blood or plasma passes through the device, the reduction or depletion of one or more targets from blood or plasma is achieved.

In preferred embodiments, an extracorporeal device is used in conjunction with an apheresis system. The apheresis process involves separation of plasma from the cellular fraction of whole blood of a subject, for example, using centrifugation, a membrane filter and/or other mechanisms. As a non-limiting example, a Spectra Optia® Apheresis System (Terumo BCT) may be used. The subject is connected to the apheresis system by obtaining vascular access, for example, using a dual lumen indwelling catheter that is placed into the jugular or carotid vein of the subject. An extracorporeal device of the present invention can be integrated into the extracorporeal circuit downstream from where the plasma is separated and into a secondary plasma device position of the circuit in accordance with the manufacturer's operating instructions for the apheresis machine. The subject's plasma is then recirculated through the extracorporeal device whereby the plasma passes through a substrate, which may comprise an adsorbent component, for the capture of one or more targets from the plasma. Flow rates can be adjusted within specific parameters that are suitable for the extracorporeal device in the circuit. The plasma is then pumped out of the system such that the treated plasma or a portion thereof is recombined with the separated cells and then is reintroduced into the circulatory system of the subject. In certain embodiments, the duration of an apheresis procedure is between 1 hour and 4 hours. In other embodiments, the treatment procedure is performed by processing between 1 and 4 plasma volumes of the subject.

In preferred embodiments, 1.5 to 3 plasma volumes are processed. In a preferred embodiment, an apheresis procedure is performed prior to delivery of a dose of chemotherapy to the subject (e.g., on the same day and/or a day prior to chemotherapy administration) and/or following chemotherapy administration (e.g., on day 1 post-drug and/or on subsequent days). Certain embodiments of the invention involve measuring the performance of an extracorporeal device during an apheresis procedure. For example, the percentage of a target removed can be determined using the initial concentration of the target in plasma prior to an apheresis procedure and its concentration at the end of the treatment session. The invention provides extracorporeal devices that remove at least 15%, 20%, 25%, 30%, 40%, 50%, 60%, 70%, 80%, 90%, 95% or more of a target during an apheresis procedure. In additional embodiments, the capture efficiency of an extracorporeal device is evaluated by assessing the concentrations of a target flowing into the device and the concentrations in the outflow from the device. The invention provides extracorporeal devices that maintain a capture efficiency of at least 15%, 20%, 25%, 30%, 40%, 50%, 60%, 70%, 80% or more capture of a target after 5, 10, 15, 20, 30, 40, 60, 75, 90, 120, 150, or 180 minutes during an apheresis procedure. The flow rate of plasma through the extracorporeal device can be adapted to maximize the efficiency of performance of the extracorporeal device. In general, lower flow rates increase the procedure time but improve the efficiency of target capture by an extracorporeal device. Exemplary flow rates that are possible with an apheresis machine are in between about 30 mL/min and up to about 300 mL/min, however, within this range, the device dimensions and other features will influence the flow rates that are allowable. In specific embodiments, an apheresis procedure is used to remove a target from plasma that comprises one or more of the following: a chemotherapy drug, a metabolite of a chemotherapy drug, an extracellular vesicle such as an exosome, a molecule or drug that is associated with or contained within an extracellular vesicle, or portions thereof.

In certain embodiments, an extracorporeal procedure (e.g., apheresis, hemodialysis, or therapeutic plasma exchange) is performed at least once prior to administration of a dose of a chemotherapy drug to a subject to achieve the desired levels of reduction of exosomes (or extracellular vesicles) from a blood component. In some embodiments, an extracorporeal procedure is performed daily, for example, every day for up to 7 days prior to administration of a chemotherapy drug, or several times per week (between 2-7 procedures), depending on the desired exosome removal. In preferred embodiments, an extracorporeal procedure may be performed prior to infusion of a chemotherapy drug or after drug administration. or both. In some embodiments, an apheresis procedure is performed at least once following administration of the chemotherapy drug to the subject for the removal of chemotherapy drug and/or exosomes. In other embodiments, an extracorporeal procedure is performed multiple times following drug delivery; for example, on multiple, successive days post-drug infusion, or at regular intervals weekly for up to 1, 2, 3, or 4 weeks. One of skill in the art would consider the established pharmacokinetics and pharmacodynamics for a particular chemotherapy drug in determining when to initiate an extracorporeal procedure post-drug dosing. This consideration would also be timed to mitigate or prevent the adverse events that accompany treatment with a particular drug. For example, in some embodiments, an extracorporeal procedure may be performed prior to the elimination half-life of the specific chemotherapy drug, which can be determined by one skill in the art for a particular chemotherapy drug. In other embodiments, an extracorporeal procedure may be performed between one day and one week or longer following administration of a dose of drug.

Aspects of the invention involve selection of the appropriate cartridge for an extracorporeal device. In certain embodiments, a cartridge comprises a housing, an inlet port that receives a blood or a blood component (e.g., plasma), a chamber or compartment configured to receive the blood component from the inlet, and an outlet port configured to pass the treated blood or blood component out of the cartridge. The housing may be formed from plastic and/or other materials; non-limiting examples include polypropylene, polystyrene, polycarbonate, or Makrolon™ polycarbonate. The end caps may be affixed or coupled to the housing. In certain embodiments, the end caps terminate at an inlet and/or an outlet. An inlet and/or an outlet may be configured to be fluidically connected to tubing sets for apheresis. The length and diameter of the housing may be selected to optimize the flow dynamics of blood or plasma through the device. A cartridge has an internal capacity that can be selected to accommodate the appropriate volume and/or surface area of the internal components. In certain embodiments, the internal volume of the cartridge is between 30 mL and 150 mL. In certain embodiments, a chamber comprises at least one substrate or adsorbent. In certain embodiments, a cartridge comprises one or more access ports or elution ports. For example, a cartridge may comprise one or more ports configured to facilitate removal of all or part of a substrate/adsorbent or a target molecule from the compartment. The portion of the cartridge that contacts the blood or plasma is sterile and suitable for medical use.

In certain embodiments, a compartment or chamber in a cartridge comprises at least one substrate that is configured to bind to at least one target from blood or plasma. In some embodiments, a substrate comprises a solid support to which an affinity reagent is attached, wherein the substrate itself does not directly bind to a target molecule. Preferably, the at least one target molecule that is bound by an affinity reagent comprises a chemotherapy drug, a metabolite of a chemotherapy drug, an extracellular vesicle such as an exosome, a molecule or drug that is associated with or contained within an extracellular vesicle, or portions thereof.

In some embodiments, a substrate provides a surface for attachment of other molecules or compounds, for example, an antibody, a Fab fragment, an aptamer, a small molecule, or another ligand. In some embodiments, a substrate comprises a bead, a membrane, a non-porous or porous particle, a porous membrane, a porous filter, or a porous monolith, or a combination of multiple structural types. In certain embodiments, a substrate comprises a biocompatible polymer, for example, a polymer comprising polyurethane, polypropylene, polysulfone, polycarbonate, polyethersulfone/polyethylene oxide copolymer, nylon, polyimide, or other synthetic resins known to those skilled in the art. A preferred polymer is polyethersulfone, owing to its chemical stability and physical strength. Specific methods for producing these polymers are known to those skilled in the art. In certain embodiments, a substrate comprising a particle or bead may be spherical, roughly spherical, or non-spherical, and may be of a size ranging from 5 microns to 5 mm. In certain embodiments, a substrate comprises an affinity chromatography support material. Embodiments of the invention disclosed devices comprising one or more of the following components or materials: agarose, Sepharose®, cellulose, xylan, dextran, pullulan, starch, pore glass, silica, acrylamide and/or its derivatives, polyacrylamide beads, trisacryl, sephacryl, ultrogel AcA, azlactone beads, methacrylate derivatives, TSK-Gel Toyopearl® HW, HEMA, Eupergit®, Poros™, polystyrene and/or its derivatives, carbon, charcoal, or combinations thereof. In certain embodiments, a substrate comprises hollow fiber membranes, sheet or rolled sheet membranes, or membrane cassettes. In certain embodiments, a substrate is modified to contain chemically active groups that can interact with a target, for example, with an extracellular vesicle or a fragment thereof. In certain embodiments, a substrate comprises a surface, a resin, or a bead to which one or more ligands, compounds, molecules, antibodies, Fab fragments, aptamers, or fragments thereof are covalently attached or otherwise functionalized to the substrate. In this aspect, the density and orientation of a ligand or another molecule on said substrate must be configured to facilitate binding to a target from blood or plasma.

In certain embodiments, a substrate comprises at least one adsorbent that binds or captures at least one target by either physical adsorption (e.g., via Van der Waals-type forces) or chemisorption (i.e., through a chemical bond). In some embodiments, an adsorbent comprises an affinity agent that interacts directly with a target to facilitate binding or capture of said target (for example, a monoclonal antibody). For example, as is known in the art, a conventional method for adsorption of an exosome employs a ligand such as a monoclonal antibody that is directed toward a specific antigenic region on an exosomal surface molecule, for example with a tetraspanin (CD9, CD63, CD81) or another molecule. In other embodiments, an adsorbent comprises a polymeric matrix, resin, bead, or another material to which a target binds or adheres directly. In preferred embodiments, an adsorbent comprises pores of heterogeneous sizes that serve as binding sites for one or more targets in blood or plasma. In certain embodiments said adsorbent comprises pores with sizes between 10 nm and 100 nm. In yet other embodiments, an adsorbent comprises pores with sizes less than 10 nm and/or greater than 100 nm. In preferred embodiments, an adsorbent is configured to bind and remove at least one target comprising a chemotherapy drug, a metabolite of a chemotherapy drug, an extracellular vesicle such as an exosome, a molecule or drug that is associated with or contained within an extracellular vesicle, or portions thereof. The invention provides adsorbent materials for treating blood or plasma of a subject with cancer, wherein targets relevant to the safety and efficacy of a chemotherapy drug can be removed. The invention satisfies the need for adsorbent materials that are useful for extracorporeal removal of toxic chemotherapy drug from the circulation including free drug as well as drug that is sequestered in and transported by exosomes.

In certain embodiments, an adsorbent is coated with albumin prior to use, for example, using human albumin. In some embodiments, the adsorbent material is directly coated with albumin, for example, as a step in a manufacturing process. In other embodiments, the adsorbent is treated with albumin during priming of the extracorporeal system. Coating of an adsorbent with albumin may be performed to increase the biocompatibility of the adsorbent.

In preferred embodiments, an adsorbent is contacted with plasma in an extracorporeal device. Disclosures herein include extracorporeal devices that are configured with internal structures that facilitate physical separation of plasma or a fraction thereof from whole blood entering the device, for example, based on size exclusion and/or filtration mechanisms. By way of example, embodiments of the invention include porous hollow fiber filters, wherein the size of the pores in the fiber walls may be appropriately sized and dimensioned to permit the passage of plasma components to access an adsorbent component (which may comprise one or more adsorbents) while the remainder of whole blood components are excluded from contacting the adsorbent. In other embodiments, plasma separation occurs in a distinctive compartment or device upstream from an extracorporeal device comprising an adsorbent component. For example, an extracorporeal circuit is disclosed by Roberts and Litzie in U.S. Pat. No. 8,038,638 B2 that provides sequential exposure of whole blood to a first device, a plasma filtration device, to obtain plasma or a plasma component, followed by exposure to a second device, an extracorporeal device for removal of one or more targets. In yet other embodiments, an extracorporeal device is used with an industry-standard apheresis machine, wherein the extracorporeal system uses centrifugal or membrane separation techniques to separate plasma from whole blood and the plasma is then directed to the extracorporeal device for removal. Examples of commercially available apheresis systems that are suitable for practicing this embodiment include but are not limited to the Terumo BCT Spectra Optia® System and the Fresenius Amicus™ System.

In some embodiments, an extracorporeal device comprises a hollow fiber membrane (which may interchangeably be referred to as a “filter”) disposed within a cartridge housing, wherein the hollow fiber membrane is comprised of a plurality of hollow fibers having fiber walls and a plurality of pores. In certain embodiments, the pores are sized and configured to allow blood or plasma components as small as 0.5 nm and as large as 200 nm to pass through the walls of the hollow fibers into an extraluminal space. In some embodiments, the hollow fiber membranes extend the length of the device. As whole blood or plasma enters the device, blood or plasma components with diameters less than 200 nm pass through the plurality of pores into an extraluminal space. In contrast, cells and blood or plasma components having diameters greater than about 200 nm in size are blocked from entering the extraluminal space and instead pass through the lumen of the hollow fibers. In preferred embodiments, the extraluminal space comprises one or a plurality of adsorbents. As whole blood or plasma is filtered through the device, the plasma components that have accessed the extraluminal space encounter the one or plurality of adsorbents that positioned inside the housing and outside the hollow fiber filter. The targets are exposed to the plurality of adsorbents in the extraluminal space such that the targets are bound, captured, sequestered, and/or adsorbed by the adsorbents, thereby reducing the amount of the target in the plasma. The device also comprises an inlet port for receiving unfiltered (or untreated) blood or plasma and an outlet port for returning the treated blood or plasma, which has been depleted of targets, back to the circulatory system of the subject. As will be appreciated by one of ordinary skill in the art, the blood or plasma entering the device is circulated to flow at rates sufficient to create pressure that will cause plasma comprising the targets to flow through the fiber walls. The blood flow rates through the hollow fibers of the device may exceed 100 mL/min but are preferably not greater than about 500 mL/min.

In some embodiments, a hollow fiber membrane has pores in the fiber walls that may range up to 500 nm in size. In certain embodiments, a hollow fiber membrane has a plurality of pores with sizes between about 20-200 nm, about 20-300 nm, about 20-400 nm, or about 20-500 nm. In some embodiments, the pores are sized to allow targets in blood or plasma as small as 0.5 nm to pass through and access the extraluminal space. In some embodiments, the pores are sized to allow targets in blood or plasma as large as 200 nm, as large as 300 nm, as large as 400 nm, or as large as 500 nm to pass through the pores in the fiber walls. Accordingly, the methods of the invention include selection of a hollow fiber membrane based on its pore size for the removal of targets having a known size. As a non-limiting example, hollow fibers with pores up to 200 nm in size or larger would be appropriately sized for selecting certain chemotherapy drugs (average sizes of 100-150 nm for nanocarrier-based drugs and smaller for other drugs) and exosomes (average sizes of 100 nm).

In specific embodiments, a device comprising a hollow fiber membrane also comprises one or more adsorbents disposed in the extraluminal space outside the hollow fibers and within the device housing. An adsorbent may comprise a membrane, resin, bead, or other material binds to one or more targets via an adsorptive mechanism of action. Embodiments of the invention therefore provide an extracorporeal device having a dual mechanism of action involving size exclusion to select blood or plasma components and subsequent adsorption of targets from the selected blood or plasma components.

Embodiments of the invention involve selecting hollow fiber membranes having certain physical and chemical specifications, including membrane material, surface contact area, and molecular size cutoff. As will be appreciated by one of skill in the art, the configuration and composition of the hollow fibers within the module can be modified to alter the performance of the device. In selecting a hollow fiber membrane, one of skill in the art also considers the blood or plasma volume to be treated and the flow rate of blood or plasma through the system. In certain embodiments, a hollow fiber can have average or median pore sizes that are approximately 100 nm, approximately 200 nm, approximately 500 nm, or larger, thereby restricting the passage of particles that are larger than the size cut-off. In non-limiting examples, hollow fiber membranes that are useful for the following invention may comprise hollow fibers with the following specifications: between 400-800 hollow fibers, surface contact areas ranging from approximately 1.0 m² to 8.0 m², a fiber length ranging from 60 mm to 300 mm. Commercially available hollow fiber membranes may be selected for use with the invention, non-limiting examples of which include Plasmart cartridges sold by Medica SPA and Plasmaflux P2 hollow fiber filters sold by Fresenius.

Certain aspects of the invention provide methods for laboratory bench testing of an adsorbent component to determine and/or quantify the binding of said adsorbent to a target such as a chemotherapy drug or an exosome. A target can be spiked at a known concentration into a solution comprising plasma or a buffer. For evaluation of exosome binding or capture by an adsorbent, one of skill in the art would find it useful to employ target particles with similar physical and biochemical properties as the intended in vivo targets. For example, liposomes comprise vesicles having lipid bilayers that are commercially available in sizes comparable to exosomes. Embodiments of the invention involve exposing of applying one or more target compounds or vesicles to an adsorbent component in vitro for a defined time period and then quantifying capture of said target(s). In other embodiments, an extracorporeal device comprising an adsorbent component is evaluated for target capture in vitro using similar steps.

In certain embodiments, an extracorporeal device reduces the quantity or concentration of a target in blood or plasma of a subject, wherein a target comprises a chemotherapy drug and/or an exosome that is reduced by at least 10%, 15%, 20%, 25%, 30%, 40%, 50%, 60%, 70%, 80%, 90%, 95% or more in a single treatment session. In another embodiment, an extracorporeal device reduces the concentration of a target in a subject to similar levels as are present in one or more control subjects, wherein a control may be a healthy individual or a pre-disease state or earlier disease stage in the same subject or in a cohort of subject(s). In certain embodiments, a “similar” level of a target in extracorporeal device treated subject(s) is defined as being within 5%, within 10%, within 20%, within 30% or within 40% of the concentration in the control subject(s).

It is the inventors' experience that an extracorporeal device comprising a plurality of adsorbents can provide beneficial effects for binding or sequestering physically and biochemically heterogeneous targets from a blood component. In some an extracorporeal device comprises a unitary structure having disposed therein one or more adsorbents, wherein the one or more adsorbents are preferably present as a mixture such as a slurry. In other embodiments, each adsorbent is placed in a discrete chamber or compartment within an extracorporeal device, wherein the chambers or compartments are connected in parallel or in series for receiving blood or plasma from a subject. In other embodiments, the extracorporeal methods are conducted using a plurality of devices, wherein the plurality of devices differ with respect to their adsorbent composition and sequential treatments with the devices are performed for treating a subject. However, certain adsorbents disclosed herein, by virtue of their immense surface areas and adsorption properties, are also useful individually for binding or sequestering one or more targets from blood or plasma.

In certain embodiments, an adsorption component comprises activated carbon, one or more non-ionic exchange resins, or combinations thereof. Specific embodiments disclose adsorbents comprising activated carbon and at least one non-ionic exchange resin. In certain embodiments, the non-ionic exchange resins comprise a non-ionic aliphatic ester resin and non-ionic polystyrene divinyl benzene resin. In preferred embodiments, the adsorbents comprise activated carbon, a non-ionic aliphatic ester resin, and a non-ionic polystyrene divinyl benzene resin. Preferably, the activated carbon, the non-ionic aliphatic ester resin, and the non-ionic polystyrene divinyl benzene resin are admixed in a slurry. The mixture of adsorbents may be disposed in a compartment or chamber of an extracorporeal device, wherein the adsorbent mixture is in contact with targets in blood or plasma during an extracorporeal procedure. In certain embodiments, the adsorbent components in the mixture are present at defined ratios, wherein a preferred ratio of activated carbon to non-ionic aliphatic ester resin to non-ionic polystyrene divinyl benzene resin is from about 10:1:1 to about 1:1:1 on weight basis.

Additional embodiments disclose adsorbents comprising an ion exchange resin. The adsorption component can comprise an ion exchange resins, non-ionic exchange resins, activated carbon, or combinations thereof.

In specific embodiments, an adsorption component is configured to bind to one or more targets by one or more of the following mechanisms: hydrophobic interactions, ionic or electrostatic interactions, hydrogen bonding, and/or van der Waals interactions. Advantageously, a combination of adsorbents facilitates binding or adsorption to a target via more than one type of interaction, thereby improving the percentage of target that is effectively captured from blood or plasma.

In certain embodiments, an adsorbent component comprising activated carbon is configured to bind or remove one or more of the following targets: a chemotherapy drug, a metabolite of a chemotherapy drug, an extracellular vesicle such as an exosome, a molecule or compound that is associated with an extracellular vesicle, or portions thereof. Activated carbon is produced by heat treatment or activation of raw materials, which creates its pore surface area and its adsorptive capabilities. Activated carbon may comprise powdered activated carbon and/or granular activated carbon. In certain embodiments, activated carbon is coated with a semi-permeable film-forming material comprising pyroxylin, polypropylene, vinyl chloride-vinylidene chloride copolymer, ethylene glycol polymethacrylate, collagen, whereas in other embodiments of the invention, the activated carbon is uncoated. Precursor materials and manufacturing methods for preparing an activated carbon resin are known to one of ordinary skill in the art. Typically, activated carbon can be produced from source materials of botanical origin for example wood, nut shells, coconut shells, or from degraded and coalified plant matter such as peat and coal. In certain non-limiting embodiments, an activated carbon adsorbent comprises coated coconut shell granule, uncoated coconut shell granule, uncoated synthetic charcoal, and/or uncoated organic granule charcoal. In certain embodiments, an adsorbent comprises a carbon nanotube, wherein a carbon nanotube may be single-walled or multi-walled.

Due to the high porosity of activated carbon, this adsorbent has an enormous surface area that makes it highly suitable for binding to targets that are present in high concentrations. Commercial activated carbon may have an internal surface area of 500 m²/g up to 1500 m²/g or even >3000 m²/g. In certain embodiments, an adsorbent comprising activated carbon has a surface area of at least 500 m²/g, 500 m²/g, 1000 m²/g, 1500 m²/g, 2000m²/g, 2500 m²/g or 3000 m²/g. In some embodiments, activated carbon is produced having a particular range and distribution of pore sizes to facilitate binding to one or more targets of relevance to the invention. In certain embodiments, activated carbon comprises a pore size distribution comprising a micropore region of less than 100 Angstroms, a mesopore region of between 100 and 1,000 Angstroms, and a macropore region with pore size diameters greater than 1,000 Angstroms.

In certain embodiments, an adsorbent component comprising a non-ionic exchange resin is configured to bind to one or more of the following targets: a chemotherapy drug, a metabolite of a chemotherapy drug, an extracellular vesicle such as an exosome, a molecule or compound that is associated with an extracellular vesicle, or portions thereof. Non-ionic exchange resins advantageously are unlikely to adsorb essential cations and anions from blood or plasma. Non-ionic exchange resins adsorb weakly polar and non-polar compounds or molecules primarily via van der Waals interactions. Specific non-limiting examples of non-ionic exchange resins that are suitable for use with the present invention include the commercially available resins, Amberchrom™ CG300-C and Amberlite™ XAD-7 HP. Amberchrom™ CG300-C resins are synthetic non-ionic polymeric resins made from polystyrene divinyl benzene. Amberlite™ non-ionic polymeric resins can have a range of particle sizes, surface areas, average porosities, and pore sizes. One of ordinary skill in the art may select an alternate or additional non-ionic exchange resin for application to the present invention.

In one aspect, a non-ionic aliphatic ester resin has an average surface area of approximately 500 m²/g, an average pore size of 300-600 Angstroms, and a mean particle diameter of approximately 560 microns.

In one aspect, a non-ionic polystyrene divinyl benzene resin has an average surface area of approximately 700 m²/g, an average pore size of 300 Angstroms, and mean particle diameter from approximately 35 microns to approximately 120 microns.

In one aspect, a non-ionic polystyrene divinyl benzene resin has an average surface area of approximately 600 m²/g, an average pore size of 100-400 Angstroms, and mean particle diameter from approximately 300 microns to approximately 500 microns.

In other embodiments, an adsorbent comprises an ion exchange resin. An ion exchange resin comprises an insoluble matrix in the form of beads that may be fabricated from an organic polymer substrate, comprising a structure of pores on the surface that allow for trapping of ions occurs with the simultaneous release of other ions. An ion exchange resin may comprise a solid phase with sites that are negatively charged (i.e., a cation exchange resin) or positively charged (i.e., an anion exchange resin). In certain embodiments, an ion exchange resin is charged through attachment of one or a plurality of ligands to the resin (e.g., by covalent linking) or, in other embodiments, the charge may be an inherent property of the solid phase (e.g., silica). Ion exchange resins may be broadly classified as strong or weak acid cation exchangers or strong and weak base anion exchangers. In certain embodiments, an adsorbent comprises an anion exchange resin for removal or capture of extracellular vesicles or exosomes from blood or plasma. This embodiment exploits the net negative charge of these vesicles by binding them to a positively charged membrane, matrix, or bead. Anion exchange materials are known to one skilled in the art and these resins may be selected from commercially available resins, non-limiting examples including QAE SEPHADEX (Cytiva) and FAST Q SEPHAROSE (Cytiva).

Certain embodiments provide an extracorporeal device for selecting and removing a chemotherapy drug from blood or plasma of a subject comprising: (a) an inlet for receiving blood or plasma from the subject, wherein said blood or plasma comprises an initial quantity of a chemotherapy drug; (b) an adsorbent comprising activated carbon and at least one non-ionic exchange resin for adsorbing and retaining a quantity of chemotherapy drug in blood or plasma, wherein said adsorbent is configured to bind to a circulating chemotherapy drug and to drug-containing exosomes; and (c) an outlet for returning blood or plasma to the subject, wherein the returned blood or plasma has a reduced quantity of chemotherapy drug as compared to the initial quantity.

Other embodiments provide an extracorporeal device for selecting and removing nanovesicles from blood or plasma of a subject undergoing chemotherapy comprising: (a) an inlet for receiving blood or plasma from the subject; (b) an adsorbent comprising activated carbon and at least one non-ionic exchange resin for adsorbing and retaining a quantity of nanovesicles from blood or plasma comprising nanovesicles, wherein said adsorbent is configured to bind to a liposomal chemotherapy drug and to drug-containing exosomes; and (c) an outlet for returning blood or plasma to the subject, wherein the returned blood or plasma has a reduced quantity of nanovesicles as compared to the initial quantity.

In certain embodiments, an extracorporeal device is provided comprising: (a) an inlet port for receiving unfiltered blood or plasma from a subject; (b) a compartment comprising a plurality of hollow fibers, wherein said hollow fibers have pore sizes of approximately 200 nm for passage of a plasma component comprising a chemotherapy drug; (c) an extraluminal space comprising an adsorbent composition, wherein the adsorbents comprise activated carbon or at least one non-ionic exchange resin or both for contacting and binding the plasma component comprising a chemotherapy drug to form filtered blood or plasma; and (d) an outlet for returning filtered blood or plasma back to the subject. In preferred embodiments, a plasma component comprises free chemotherapy drug and/or exosome-associated chemotherapy drug. In additional embodiments, the chemotherapy drug comprises a liposomal drug formulation.

The adsorbent components disclosed herein are well-suited for reduction of extracellular vesicle populations that are present in blood or plasma. The inventors have determined that a composition of activated carbon and non-ionic exchange resins as adsorbents can effectively capture nanovesicles (i.e., liposomes) from a fluid. Liposomal drugs, other nanocarrier-based drugs and exosomes have similar sizes and membrane compositions as other liposomes. By way of example, the average diameter of an endogenous exosome in blood is approximately 100 nm, liposomal doxorubicin is approximately 100 nm in size, and abraxane (albumin-bound paclitaxel in a colloidal suspension) is approximately 130 nm in size. Accordingly, in certain embodiments, the extracorporeal devices disclosed herein are configured to bind targets between about 20 nm to 1,000 nm in diameter. In other embodiments, the extracorporeal devices are configured to bind targets less than 100 nm, 150 nm, 200 nm, 300 nm, 500 nm, or less than 1,000 nm in diameter. The adsorbent compositions disclosed herein may provide advantages over established methods for capturing exosomes from biofluids, particularly considering the abundance of exosomes (up to 10¹² vesicles/mL of plasma/serum or higher). The present invention has the merit of avoiding the need for costly ligands as adsorbents while providing an expansive adsorption interface that is required for contacting the entire plasma volume.

In other embodiments, an adsorbent comprises a lectin bind to a surface glycan (i.e., a polysaccharide) present on the membrane surface of an extracellular vesicle or an exosome in blood or a blood component. In the context of the invention, a lectin may comprise an affinity agent for the binding and removal of an exosome from blood or a blood component using an extracorporeal device. It is contemplated that specific lectins bind to glycans that are highly expressed by cancer cells and, correspondingly, bind to exosomes that are packaged and released by said cancer cells. In certain embodiments, a lectin binds to one or more of the following moieties or residues on an exosome: sialic acid, O-glycan (e.g., Galα1-3GalNAc), mannose, complex N-linked glycan, or poly N-acetyllactosamine. In specific embodiments, a lectin comprises one or more of the following: sialic acid-binding immunoglobulin-like lectin, Sambucus nigra lectin (SNA), Maackia amurensis lectin (MAL), Amarylis lectin (HHL), Pisum sativum agglutinin (PSA), Narcissus pseudonarcissus agglutinin (NPA), Galanthus nivalis agglutinin (GNA), Scytovirin (SVN), Urtica dioica agglutinin (UDA), Calystegia sepium lectin (Calsepa), Lens culinaris (LcH), Phaseolus vulgaris-E (PHA-E), Phaseolus vulgaris-L (PHA-L), Tulipa sp., Trichosanthes japonica (TJA-I, TJA-II), Lypersicon esculentum (LEA), Datura stramonium agglutinin (DSA), Solanum tuberosum lectin (STL), Lycopersicon esculentum lectin (LEL), Cyanovirin N (CVN), Amaranthus caudatus agglutinin (ACA), and Agaricus bisporus agglutinin (ABA). Embodiments of the invention disclose an extracorporeal device comprising a lectin, wherein said lectin binds to an exosome or a portion thereof present in blood or a blood component.

Additional embodiments of the invention contemplate mixing or attaching additional adsorbent compounds or materials into an extracorporeal device for refining or increasing the scope of drug or molecular targets that are adsorbed. As a non-limiting example, three-dimensional nanostructures can be functionalized to one or more of the substrates disclosed herein. Exemplary embodiments are nanostructures that are applicable as drug carriers that are biocompatible, highly stable, and can be unmodified or modified by attaching ligands or other moieties. Examples of nanostructures for use in the present invention are tetrahedral DNA nanostructures, which are useful for capturing DNA-binding chemotherapy drugs (e.g., alkylating agents). In preferred embodiments, an adsorbent comprises a tetrahedral DNA nanostructure binds to a chemotherapy drug (e.g., doxorubicin). As another non-limiting example, a ligand for highly selective binding to a target such as an exosome can be functionalized to an adsorbent of the invention; for example, one or more antibodies directed against exosomal surface proteins such as tetraspanins (CD9, CD63, CD81) or fusion proteins (e.g., flotillin and annexin). Embodiments of the invention include measuring the effectiveness of an adsorbent composition for binding or selecting an extracellular vesicle or an exosome from a fluid (e.g., blood or plasma) or an appropriate solvent or buffer in vitro. Laboratory testing may utilize a target that is spiked into a fluid or solvent at a known concentration. In other embodiments, assessment of an adsorbent composition utilizes a blood or plasma specimen derived from a subject, wherein endogenous concentrations of a target are present. In certain embodiments, a procedure for evaluating an adsorbent composition in an extracorporeal device employs a pump for driving the flow of the solution or specimen through the inlet of the device, through a compartment or chamber comprising the adsorbent composition, and then out of the device via an outlet, for a predetermined length of time (for example, for at least 15 min, 30 min, 1 hour, 2 hours, 3 hours, or longer). A pump may comprise a peristaltic pump, a syringe pump, a piston pump, and/other pumps, which control the flow rate of solution through the device. In other embodiments, an adsorbent is provided as a solution or slurry that is exposed to a target in vitro for a defined duration (e.g., 15 min, 30 min, 1 hour, or 2 hours). In some embodiments, the concentration of a target remaining in solution at a defined time point in the laboratory procedure is assessed relative to its concentration in the starting fluid or solution, which may be calculated as the percentage of the target removed. In certain embodiments, the percentage of target removed by an adsorbent component is at least 10%, 15%, 20%, 25%, 30%, 40%, 50%, 60%, 70%, 80%, 90%, 95% or more.

### EXAMPLE

Some aspects of the embodiments discussed above are disclosed in further detail in the following non-limiting example.

This example provides pre-clinical data on the performance of an adsorbent composition for binding liposomes. In addition to their application as nanoparticle delivery systems for chemotherapeutic drugs, liposomes are an established model system for studying extracellular vesicles and their exosome subsets. The in vitro system therefore serves as a model for the removal of nanovesicles, including liposomal drugs as well as endogenous exosomes from blood or plasma using a particular combination of adsorbents.

In this example, the adsorbent preparation comprised a mixture of activated carbon and two non-ionic exchange resins, Amberchrom™ CG300-C and Amberlite™ XAD-7 HP, which are a polystyrene divinyl benzene resin and an aliphatic ester resin, respectively. The experiment measured the binding of this adsorbent preparation to liposomes.

In pre-clinical testing, a sorbent slurry was used in pilot studies to assess the potential of the sorbent system to remove larger molecular weight targets such as extracellular vesicles or exosomes. Fluorescent liposomes (˜100 nm) were chosen as a model analog for each of these targets. There is ample literature supporting the use of liposomes with lipid and cholesterol constituents as a model system to evaluate the extracorporeal clearance of microvesicular particles, extracellular vesicles or exosomes.

Sorbent slurry preparation: In the pre-clinical testing studies, a specified sorbent admixture (Table 1) was prepared by wetting with 70% ethanol, and collection using a 10-micron filter and Buchner funnel setup with the aid of vacuum.

TABLE 1
Sorbent admixture used for rocker studies

| Sorbent Component | Amount (g) |
|---|---|
| Activated Carbon | 70.7 |
| Amberlite | 28.3 |
| Amberchrom | 28.3 |
| Total | 126.3 |

Sorbents were suspended in ˜250 mL of solvent, which resulted in a total apparent volume of ˜380 mL. Well-mixed sorbent slurry was poured onto filter paper, and excess solvent was removed via vacuum setup. Sorbent was weighed out (1.00±0.05 g or 2.00±0.10 g as applicable) and added to test tubes.

Rocker study format: Test tubes filled with sorbent and target liposomes were rocked on a nutator rocker (multidirectional tilting rocker) at room temperature for 2 hrs. Test tubes were centrifuged for a minimum of 1,500×g for an at least 10 minutes, to allow for the separation of plasma/target mixture from sorbent. In most cases, even after 10 minutes of centrifugation, supernatant was still a bit cloudy with some fine carbon particles, but otherwise appeared free of sorbent constituents. Full removal of carbon from supernatant was not attained with subsequent spins. Carbon-free zones of supernatant were sampled as much as possible as aliquots for fluorescent, absorbance or ELISA analysis on an M5 Spectrophotometer.

Liposome Preparation: Fluorescent liposomes (Formumax F60103F-R, 100 nm, Rhodamine labeled) were prepared at an initial concentration of ˜0.5 mM, in filtered human plasma. Of this stock, 2 mL of human plasma with liposomes was mixed with 2 g of the Sorbent slurry (prepared per ratio and filtered using a 0.2 micron filter) in a 5 mL capacity test tube. A small aliquot of initial liposome stock solution in plasma was also retained to confirm initial fluorescence.

M5 Spectrophotometer analysis of liposomes: A Rhodamine-labeled liposome standard curve was generated for 100 μL stock liposome samples (halving dilution series starting at 5 mM) loaded into a blackout plate, with fluorescence excitation at 560 nm, and emission at 580 nm, with an auto-cutoff value of 570 nm. Initial liposome/plasma solution was shown to have a starting concentration of 0.572 mM. Minimum detection threshold for this assay was 0.019 mM.

Liposomes: All values were measurable based on the fluorescence standard ladder. Initial liposome/plasma solution was shown to have a starting concentration of 0.572 mM, which dropped to a value of 0.043 mM in 2 hrs, suggesting a liposome reduction of 92.5% with sorbent exposure.

Discussion: Comparison of initial and final concentrations of liposomal targets (a model for extracellular vesicles and exosomes ˜100 nm in size) demonstrated a >92% reduction by this sorbent formulation in 2 hours. The data demonstrate the efficiency with which the adsorbent composition removes liposomes, serving as a model for the capture of exosomes as well as liposomal drug formulations by an extracorporeal device comprising these adsorbents. Based on these data, methods applying these adsorbents would be useful for an extracorporeal removal of exosomes prior to chemotherapy drug dosing as well as for depletion of exosomes and a drug post-chemotherapy administration.

What is claimed is:

1. A method for improving the safety and efficacy of a chemotherapy drug in a subject in need thereof, comprising:
a) introducing blood or plasma from a subject into a first extracorporeal device comprising an adsorbent, wherein the blood or plasma comprises an amount of a pre-chemotherapy target molecule or compound, and wherein said pre-chemotherapy target molecule or compound is induced by a tumor;
b) contacting the blood or plasma with the adsorbent in the first extracorporeal device to allow the pre-chemotherapy target molecule or compound to bind to the adsorbent;
c) reintroducing the blood or plasma into the subject, wherein the blood or plasma obtained after (b) has a reduced amount of the pre-chemotherapy target molecule or compound as compared to the blood or plasma of the subject prior to (b);
d) administering a chemotherapy drug to the subject;
e) introducing blood or plasma from the subject into a second extracorporeal device comprising a second adsorbent, wherein the blood or plasma comprises an amount of a post-chemotherapy target molecule or compound,
f) contacting the blood or plasma with the second adsorbent in the second extracorporeal device to allow the post-chemotherapy target molecule or compound present in blood or plasma to bind to the adsorbent; and
g) reintroducing the blood or plasma into the subject, wherein the blood or plasma obtained after (f) is measured to have a reduced amount of the post-chemotherapy target molecule or compound as compared to the blood or plasma of the subject prior to (f).

2. The method of claim 1, wherein the pre-chemotherapy target or compound to be bound in the first adsorbent is an exosome.

3. The method of claim 2, wherein the amount of said exosome in blood or plasma is measured by identifying expression of one or more molecules comprising: CD9, CD63, CD81, HSP70 Lamp2b, Tsg101, flotillin and/or annexin.

4. The method of claim 1, wherein the post-chemotherapy target molecule or compound that binds to the second adsorbent is selected from the group consisting of an exosome, chemotherapy drug, a metabolite of said chemotherapy drug, and combinations thereof.

5. The method of claim 1, wherein the first adsorbent and second adsorbent are the same.

6. The method of claim 1, wherein the first adsorbent comprises activated carbon.

7. The method of claim 6, wherein the activated carbon is selected from the group consisting of coated coconut shell granule, uncoated coconut shell granule, synthetic charcoal, and combinations thereof.

8. The method of claim 6, wherein the activated carbon has a pore size distribution of a micropore region of less than 100 Angstroms, a mesopore region of between 100 and 1000 Angstroms, and a macropore region of greater than 1000 Angstroms.

9. The method of claim 1, wherein the first adsorbent comprises at least one non-ionic exchange resin.

10. The method of claim 9, wherein the non-ionic exchange resin comprises a non-ionic aliphatic ester resin, a non-ionic polystyrene divinyl benzene resin, or combinations thereof.

11. The method of claim 9, wherein the non-ionic aliphatic ester resin has an average surface area of approximately 500 m²/g, an average pore size of approximately 300-600 Angstroms, and a mean particle diameter of 560 microns.

12. The method of claim 9, wherein the non-ionic polystyrene divinyl benzene resin has an average surface area of approximately 700 m²/g, an average pore size of 300 Angstroms, and a mean particle diameter from approximately 35 microns to approximately 120 microns.

13. The method of claim 9, wherein the non-ionic polystyrene divinyl benzene resin has an average surface area of approximately 600 m²/g, an average pore size of 100-400 Angstroms, and a mean particle diameter from approximately 300 microns to 500 microns.

14. The method of claim 1, wherein the second adsorbent is selected from the group consisting of activated carbon, a non-ionic aliphatic ester resin, and a non-ionic polystyrene divinyl benzene resin.

15. The method of claim 14, wherein the ratio of activated carbon to non-ionic aliphatic ester resin to non-ionic polystyrene divinyl benzene resin is from about 10:1:1 to about 1:1:1 on weight basis.

16. The method of claim 1, wherein the second adsorbent comprises at least one ion exchange resin.

17. The method of claim 16, wherein the at least one ion exchange resin comprises an anion exchange resin.

18. The method of claim 1, wherein said chemotherapy drug is delivered within a nanocarrier.

19. The method of claim 18, wherein the nanocarrier is a liposome or an exosome.

20. The method of claim 19, wherein the liposome comprises liposomal doxorubicin.

21. The method of claim 1, further comprising administering an anticoagulant selected from the group consisting of unfractionated heparin, low molecular weight heparin, citrate, and thrombin inhibitors.

22. The method of claim 1, wherein the first and second extracorporeal devices comprise hollow fiber plasma filters.

23. The method of claim 22, wherein the hollow fiber plasma filters comprise hollow fibers with pore sizes less than 500 nm.

24. The method of claim 22, wherein the first and second adsorbents are positioned outside the hollow fibers in the extraluminal space.

25. The method of claim 1, wherein the first and second extracorporeal devices are connected with blood processing systems selected from the group consisting of: hemodialysis, apheresis, continuous renal replacement therapy, and therapeutic plasma exchange (TPE).

26. The method of claim 4, wherein a chemotherapy drug is selected from the group consisting of an antimetabolite, an antimicrotubular agent, an antibiotic, an alkylating agent, an anthracycline an antibody-drug conjugate, and metabolites and combinations thereof.

27. A method for improving the safety and efficacy of a chemotherapy drug in a subject in need thereof prior to administration of a chemotherapeutic agent, comprising:
a) providing a pre-chemotherapy extracorporeal device comprising an adsorbent;
b) introducing blood or plasma from a subject into said extracorporeal device, wherein the blood or plasma comprises an amount of a target molecule or compound, and wherein said target molecule or compound is induced by a tumor;
c) contacting the blood or plasma with the adsorbent in the extracorporeal device to allow the target molecule or compound to bind to the adsorbent;
d) reintroducing the blood or plasma into the subject, wherein the blood or plasma obtained after (c) has a reduced amount of the target molecule or compound as compared to the blood or plasma of the subject prior to (c); and
e) administering a chemotherapy drug to the subject.

28. A method for improving the safety and efficacy of a chemotherapy drug in a subject in need thereof after a chemotherapeutic has been administered, comprising:
a) introducing blood or plasma from the subject into an extracorporeal device comprising an adsorbent, wherein the blood or plasma comprises an amount of the target molecule or compound,
b) contacting the blood or plasma with the adsorbent in the extracorporeal device to allow the target molecule or compound present in blood or plasma to bind to the adsorbent; and
c) reintroducing the blood or plasma into the subject, wherein the blood or plasma obtained after (b) is measured to have a reduced amount of the target molecule or compound as compared to the blood or plasma of the subject prior to (b);
wherein the extracorporeal device is configured to capture circulating target molecules or compounds from blood or plasma after chemotherapy treatment is concluded.
